(19) United States
(12) Patent Application Publication
(10) Pub. No.: US 2003/0023082 A1
(43) Pub. Date: Jan. 30, 2003

(54) Epothilone derivatives and methods for making and using the same

(76) Inventors: Gary Ashley, Alameda, CA (US); Brian Metcalf, Moraga, CA (US)

Correspondence Address: Carolyn A. Favorito, Morrison & Foerster LLP, Suite 500, 3811 Valley Centre Drive, San Diego, CA, 92130-2332, US

(21) Appl. No.: 10/145,405

(22) Filed: May 13, 2002

Related U.S. Application Data
(60) Provisional application No. 60/291,242, filed on May 15, 2001.
(60) Provisional application No. 60/309,099, filed on Jul. 31, 2001.

(51) Int. Cl. C07D 498/02; C07D 491/02

(52) U.S. Cl. 540/461; 546/115; 548/207; 548/242; 548/360.5; 544/278

(57) ABSTRACT

The present invention relates to bicyclic compounds wherein one of the cyclic moieties is a 16-membered macrocycle and to methods for making and using these compounds. The present invention provides compounds of the formula

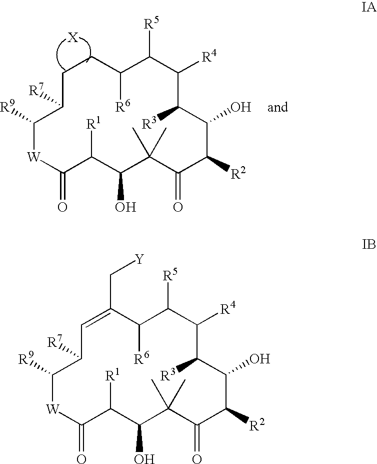

wherein:

R¹, R², and R³ are each independently hydrogen, methyl or ethyl;

R⁴ is hydrogen, hydroxyl, fluoro, oxo, oximino, or NRR′ where R and R′ are independently hydrogen, C₁-C₁₀ aliphatic, aryl or alkylaryl;

R⁵ is hydrogen, oxo, or C₁-C₁₀ aliphatic;

R⁶ is hydrogen, hydroxyl, oxo, C₁-C₁₀ aliphatic, C₁-C₁₀ alkylester, or halide, or optionally R⁵ and R⁶ together form a carbon-carbon bond;

R⁷ is hydrogen, methyl, ethyl, methoxy, or OH;

X is fused substituted or unsubstituted heterocyclo;

Y is substituted or unsubstituted heterocyclo;

R⁹ is aryl or —CH═C(Me)-Aryl; and,

W is O or NR⁸ where R⁸ is hydrogen, C₁-C₁₀ aliphatic, aryl or alkylaryl.

This applications asserts priority to U.S. Provisional Application No. 60/291,242 filed May 15, 2001, and No. 60/309,099 filed Jul. 31, 2001, both by inventors Gary Ashley and Brian Metcalf entitled EPOTHILONE DERIVATIVES AND METHODS FOR MAKING AND USING THE SAME, each of which is incorporated herein by reference in their entireties.

### BACKGROUND

Epothilone A (R═H) and Epothilone B (R═CH₃) are produced by Sorangium cellulosum strain So ce 90, the structures of which are shown below, and were the first of several epothilones to be isolated and characterized. Höfle et al., 1996, Angew. Chem. Int. Ed. Engl. 35(13/14): 1567-1569.

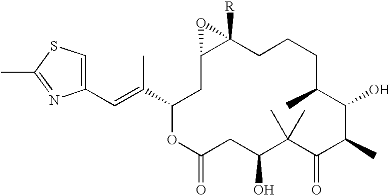

Epothilone A and epothilone B possess many of the advantageous properties of taxol. As a result, there is significant interest in these and structurally related compounds as potential chemotherapeutic agents. The desoxy counterparts of epothilones A and B are known as epothilone C(R═H) and epothilone D (R═CH₃), and also exhibit similar anti-tumor activity but with less cytotoxicity. The structures of epothilones C and D are shown below.

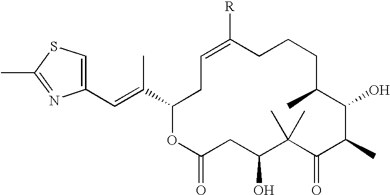

Although other naturally occurring epothilones have been described in the literature, these compounds are produced in exceedingly small amounts. For example, PCT publication WO 99/65913 describes 39 naturally occurring epothilones obtained from Sorangium cellulosum So ce 90 of which epothilones A, B, C, and D together account for approximately 98.9% of the total epothilones produced. The 35 other naturally occurring epothilone compounds together account for the remaining 1.1%.

Synthetic analogs of the natural epothilones have been reported. Glunz et al. (1999) Tetrahedron Letters 40: 6895-6898, discloses the synthesis of 12,13-benzodesoxyepothilone B, an analog having a benzene ring fused to the 12,13-bond of epothilone D. Vite et al., PCT publication WO 99/54319, discloses derivatives of epothilone C having heterocyclic groups fused to the 12,13-bond. Danishefsky et al., U.S. Pat. No. 6,204,388 discloses 26-(1,3-dioxolan-1-yl)epothilone D. Each of the above references is incorporated herein by reference.

Due to the increasing interest in epothilones as anti-cancer agents, novel derivatives of these compounds are needed and desired to more fully develop their therapeutic potential.

### SUMMARY

The present invention relates to bicyclic compounds wherein one of the cyclic moieties is a 16-membered macrocycle and to methods for making and using these compounds. In one aspect, the present invention provides compounds of the formula

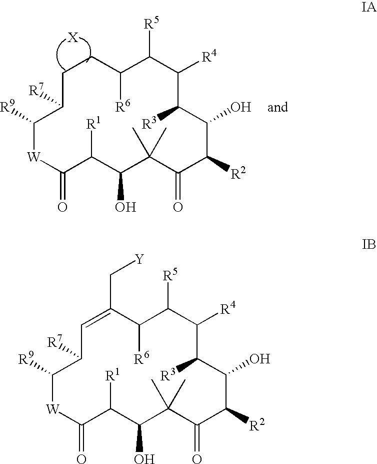

wherein:

R¹, R², and R³ are each independently hydrogen, methyl or ethyl;

R⁴ is hydrogen, hydroxyl, fluoro, oxo, oximino, or NRR′ where R and R′ are independently hydrogen, C₁-C₁₀ aliphatic, aryl or alkylaryl;

R⁵ is hydrogen, oxo, or C₁-C₁₀ aliphatic;

R⁶ is hydrogen, hydroxyl, oxo, C₁-C₁₀ aliphatic, C₁-C₁₀ alkylester, or halide, or optionally R⁵ and R⁶ together form a carbon-carbon bond;

R⁷ is hydrogen, methyl, ethyl, methoxy, or OH;

X is fused substituted or unsubstituted heterocyclo;

Y is substituted or unsubstituted heterocyclo;

R⁹ is aryl or —CH═C(Me)-Aryl; and,

W is O or NR⁸ where R⁸ is hydrogen, C₁-C₁₀ aliphatic, aryl or alkylaryl.

In a second aspect of the invention, methods for the synthesis of the compounds of formula IA and IB are provided, as well as intermediates thereto.

In a third aspect of the invention, methods for the use of the compounds of formulas IA and IB to treat cancer and non-cancer disorders characterized by undesired cellular hyperproliferation are provided.

### DETAILED DESCRIPTION OF THE PREFERRED EMBODIMENTS

The present invention relates to novel compounds that are useful for the treatment of cancer and other conditions characterized by abnormal cellular proliferation in a subject in need thereof. The present invention also relates to methods for making and using these compounds.

Definitions

Statements regarding the scope of the present invention and definitions of terms used herein are listed below. The definitions apply to the terms as they are used throughout this specification, unless otherwise limited in specific instances, either individually or as part of a larger group.

Unless explicitly indicated otherwise, all stereoisomers of the inventive compounds are included within the scope of the invention, as pure compounds as well as mixtures thereof. Furthermore, some of the crystalline forms for the compounds may exist as polymorphs and as such are included in the present invention. In addition, some of the compounds may form solvates with water (i.e., hydrates) or common organic solvents, and such solvates are also encompassed within the scope of this invention.

Protected forms of the inventive compounds are included within the scope of the present invention. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, a hydroxy protected form of the inventive compounds are those where at least one of the hydroxyl groups is protected by a hydroxy protecting group. Illustrative hydroxyl protecting groups include but not limited to tetrahydropyranyl; benzyl; methylthiomethyl; ethylthiomethyl; pivaloyl; phenylsulfonyl; triphenylmethyl; trisubstituted silyl such as trimethyl silyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl and the like; acyl and aroyl such as acetyl, pivaloylbenzoyl, 4-methoxybenzoyl, 4-nitrobenzoyl and aliphatic acylaryl and the like. Keto groups in the inventive compounds may similarly be protected.

The present invention includes within its scope prodrugs of the compounds of this invention. In general, such prodrugs are functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof Conventional procedures for the selection and preparation of suitable prodrug derivatives are described, for example, in “Design of Prodrugs”, H. Bundgaard ed., Elsevier, 1985.

As used herein, the term “aliphatic” refers to saturated and nonaromatic unsaturated straight chained, branched chain, cyclic, or polycyclic hydrocarbons that may be optionally substituted at one or more positions. Illustrative examples of aliphatic groups include alkyl, alkenyl, alkynyl, cycloalkyl, cycloalkenyl, and cycloalkynyl moieties. The term “alkyl” refers to straight or branched chain saturated hydrocarbon substituent. “Alkenyl” refers to a straight or branched chain hydrocarbon substituent with at least one carbon-carbon double bond. “Alkynyl” refers to a straight or branched chain hydrocarbon substituent with at least one carbon-carbon triple bound.

The term “cyclic aliphatic” refers to the subset of aliphatic compounds that are cyclic or polycyclic hydrocarbons. Cyclic aliphatics may be saturated or unsaturated and may be optionally substituted at one or more positions. Illustrative examples include cycloalkyl, cycloalkenyl (having one or more double bonds), cycloalkynyl (having one or more triple bonds) and cyclo-compounds having any combination of single, double and triple bonds.

The term “aryl” refers to monocyclic or polycyclic groups having at least one aromatic ring structure that optionally include one ore more heteroatoms and preferably include three to fourteen carbon atoms. Aryl substituents may optionally be substituted at one or more positions. Illustrative examples of aryl groups include but are not limited to: furanyl, imidazolyl, indanyl, indenyl, indolyl, isooxazolyl, isoquinolinyl, naphthyl, oxazolyl, oxadiazolyl, phenyl, pyrazinyl, pyridyl, pyrimidinyl, pyrrolyl, pyrazolyl, quinolyl, quinoxalyl, tetrahydronaphthyl, tetrazolyl, thiazolyl, thienyl, and the like.

The aliphatic (i.e., alkyl, alkenyl, etc.) and aryl moieties maybe optionally substituted with one or more substituents, preferably from one to five substituents, more preferably from one to three substituents, and most preferably from one to two substituents. The definition of any substituent or variable at a particular location in a molecule is independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein. Examples of suitable substituents include but are not limited to: alkyl, alkenyl, alkynyl, aryl, halo; trifluoromethyl; trifluoromethoxy; hydroxy; alkoxy; cycloalkoxy; heterocyclooxy; oxo; alkanoyl (—C(═O)-alkyl which is also referred to as “acyl”)); aryloxy; alkanoyloxy; amino; alkylamino; arylamino; aralkylamino; cycloalkylamino; heterocycloamino; disubstituted amines in which the two amino substituents are selected from alkyl, aryl, or aralkyl; alkanoylamino; aroylamino; aralkanoylamino; substituted alkanoylamino; substituted arylamino; substituted aralkanoylamino; thiol; alkylthio; arylthio; aralkylthio; cycloalkylthio; heterocyclothio; alkylthiono; arylthiono; aralkylthiono; alkylsulfonyl; arylsulfonyl; aralkylsulfonyl; sulfonamido (e.g., SO₂NH₂); substituted sulfonamido; nitro; cyano; carboxy; carbamyl (e.g., CONH₂); substituted carbamyl (e.g., —C(═O)NRR′ where R and R′ are each independently hydrogen, alkyl, aryl, aralkyl and the like); alkoxycarbonyl, aryl, substituted aryl, guanidino, and heterocyclo such as indoyl, imidazolyl, furyl, thienyl, thiazolyl, pyrrolidyl, pyridyl, pyrimidyl and the like. Where applicable, the substituent may be further substituted such as with, alkyl, alkoxy, aryl, aralkyl, halogen, hydroxy and the like.

The terms “alkylaryl” or “arylalkyl” refer to an aryl group with an aliphatic substituent that is bonded to the compound through the aliphatic group. An illustrative example of an alkylaryl or arylalkyl group is benzyl, a phenyl with a methyl group that is bonded to the compound through the methyl group (—CH₂Ph where Ph is phenyl).

The term “acyl” refers to —C(═O)R where R is an aliphatic group, preferably a C₁-C₆ moiety. The term “alkoxy” refers to —OR wherein O is oxygen and R is an aliphatic group. The term “alkylester” refers to —OC(═O)R where R is an aliphatic group. The term “aminoalkyl” refers to —RNH₂ where R is an aliphatic moiety. The terms “halogen,” “halo”, or “halide” refer to fluorine, chlorine, bromine and iodine. The term “haloalkyl” refers to —RX where R is an aliphatic moiety and X is one or more halogens. The term “heterocyclo” refers to a cyclic aliphatic that includes one or more heteroatoms. The term includes cyclic moieties having one or more double or triple bonds and includes heteroaryl groups. The term “hydroxyalkyl” refers to —ROH where R is an aliphatic moiety. The term “oxo” refers to a carbonyl oxygen (═O).

In addition to the explicit substitutions at the above-described groups, the inventive compounds may include other substitutions where applicable. For example, the lactone or lactam backbone or backbone substituents may be additionally substituted (e.g., by replacing one of the hydrogens or by derivatizing a non-hydrogen group) with one or more substituents such as C₁-C₅ aliphatic, C₁-C₅ alkoxy, aryl, or a functional group. Illustrative examples of suitable functional groups include but are not limited to: acetal, alcohol, aldehyde, amide, amine, boronate, carbamate, carboalkoxy, carbonate, carbodiimide, carboxylic acid, cyanohydrin, disulfide, enamine, ester, ether, halogen, hydrazide, hydrazone, imide, imido, imine, isocyanate, ketal, ketone, nitro, oxime, phosphine, phosphonate, phosphonic acid, quaternary ammonium, sulfenyl, sulfide, sulfone, sulfonic acid, thiol, and the like.

The term “purified” as used herein to refer to a compound of the present invention, means that the compound is in a preparation in which the compound forms a major component of the composition, such as constituting about 50%, about 60%, about 70%, about 80%, about 90%, about 95% or more by weight of the components in the composition.

The term “subject” as used herein, refers to an animal, preferably a mammal, that has been the object of treatment, observation or experiment and most preferably a human who has been the object of treatment and/or observation.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable salt” is a salt of one or more of the inventive compounds. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

The term “pharmaceutically acceptable carrier” is a medium that is used to prepare a desired dosage form of the inventive compound. A pharmaceutically acceptable carrier includes solvents, diluents, or other liquid vehicle; dispersion or suspension aids; surface active agents; isotonic agents; thickening or emulsifying agents, preservatives; solid binders; lubricants and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe, ed. (Amer. Pharmaceutical Assoc. 2000), both of which are incorporated herein by reference in their entireties, disclose various carriers used in formulating pharmaceutical compositions and known techniques for the preparation thereof.

The term “pharmaceutically acceptable ester” is an ester that hydrolzyes in vivo into a compound of the present invention or a salt thereof. Illustrative examples of suitable ester groups include, for example, those derived from pharmaceutically acceptable aliphatic carboxylic acids such as formates, acetates, propionates, butyrates, acrylates, and ethylsuccinates.

Compounds of the Present Invention

In one aspect, the present invention provides compounds of the formula

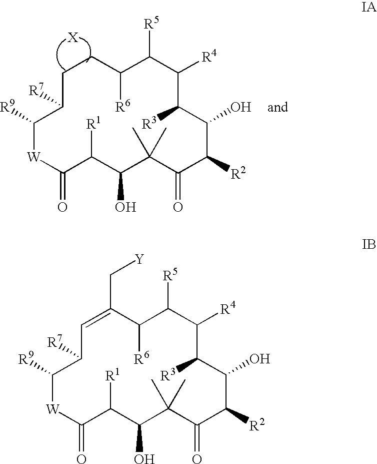

wherein:

R¹, R², and R³ are each independently hydrogen, methyl or ethyl;

R⁴ is hydrogen, hydroxyl, fluoro, oxo, oximino, or NRR′ where R and R′ are independently hydrogen, C₁-C₁₀ aliphatic, aryl or alkylaryl;

R⁵ is hydrogen, oxo, or C₁-C₁₀ aliphatic;

R⁶ is hydrogen, hydroxyl, oxo, C₁-C₁₀ aliphatic, C₁-C₁₀ alkylester, or halide, or optionally R⁵ and R⁶ together form a carbon-carbon bond;

R⁷ is hydrogen, methyl, ethyl, methoxy, or OH;

X is fused substituted or unsubstituted heterocyclo;

Y is substituted or unsubstituted heterocyclo;

Ar is aryl or —CH═C(Me)-Aryl; and,

W is O or NR⁸ where R⁸ is hydrogen, C₁-C₁₀ aliphatic, aryl or alkylaryl.

In one embodiment of the invention, compounds of the formula IIA

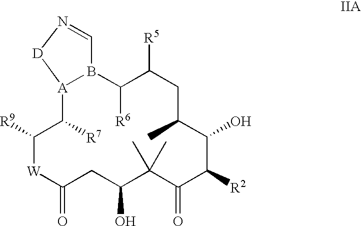

are provided wherein

R² is hydrogen, methyl or ethyl;

R⁵ is hydrogen, oxo, or C₁-C₁₀ aliphatic;

R⁶ is hydrogen, hydroxyl, oxo, or halide, or R⁵ and R⁶ together form a carbon-carbon bond;

R⁷ is hydrogen, methyl, ethyl, methoxy, or hydroxyl;

R⁹ is aryl or —CH═C(Me)-Aryl;

A—B is CH—CH or C═C;

D is O, S, NR¹⁰, CR¹⁰═N, C(OR¹⁰)═N, C(NR¹OR¹¹)═N, C(SR¹⁰)═N, C(═O)—NH, or C(═NR¹⁰)—NH, where R¹⁰ and R¹¹ are each independently H or C₁-C₅ aliphatic or aryl; and

W is O or NR⁸ where R⁸ is hydrogen or C₁-C₅ aliphatic.

In another embodiment of the invention, compounds of the formula IIA are provided wherein:

R² is methyl or ethyl;

R⁵ and R⁶ are hydrogen, or R⁵ and R⁶ together form a carbon-carbon bond;

R⁷ is hydrogen, methyl, ethyl, methoxy, or OH;

A—B is CH—CH or C═C;

D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C₁-C₅ aliphatic or aryl;

R⁹ is selected from the group consisting of

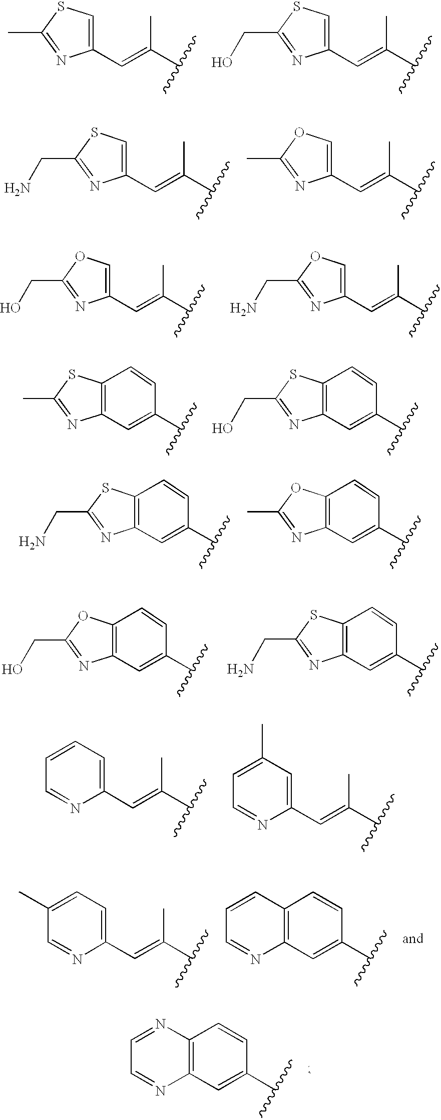

W is O or NH.

In another embodiment of the present invention, compounds of the formula IIIA are provided

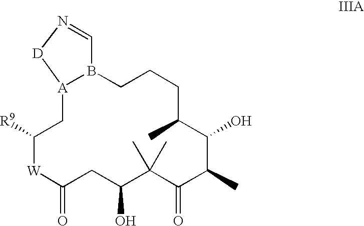

wherein:

A—B is CH—CH or C═C;

D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl;

R⁹ is selected from the group consisting of

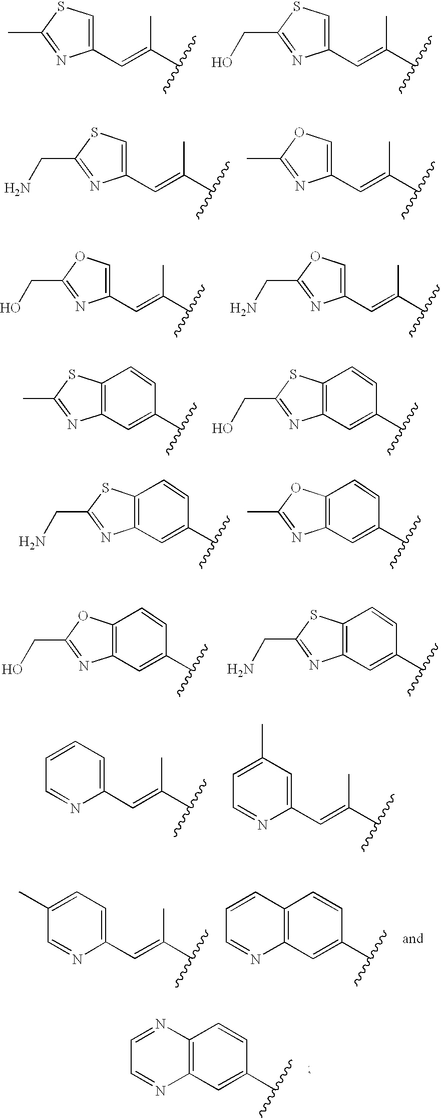

W is O or NH.

In another embodiment of the invention, compounds of formula IIIA are provided wherein:

A—B is CH—CH or C═C;

D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl;

R⁹ is

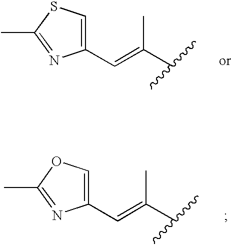

and

W is O or NH.

In another embodiment of the invention, compounds of formula IIIA having the formulas

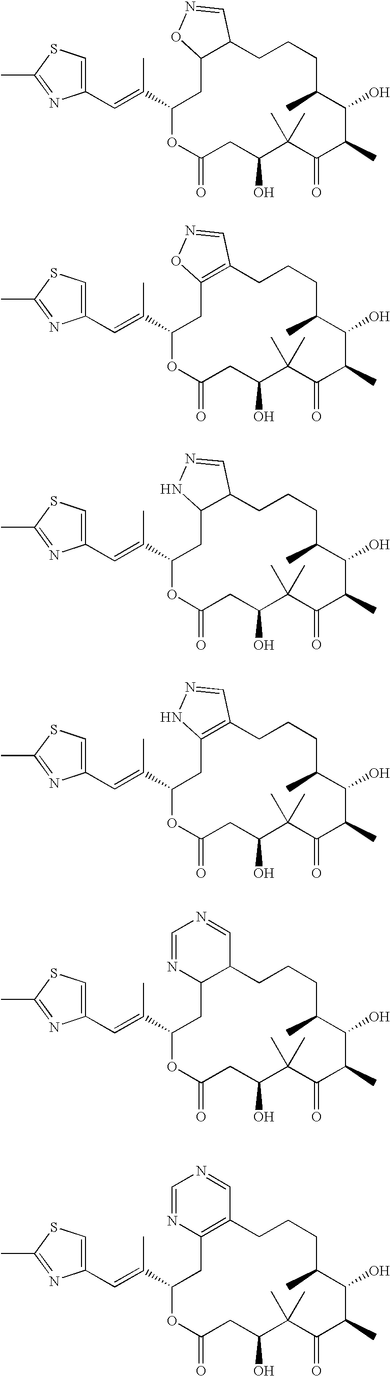

In another embodiment of the invention, compounds of formula IIB are provided

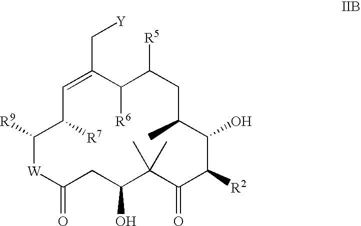

wherein

R² is hydrogen, methyl or ethyl;

R⁵ is hydrogen, oxo, or C₁-C₁₀ aliphatic;

R⁶ is hydrogen, hydroxyl, oxo, or halide, or R⁵ and R⁶ together form a carbon-carbon bond;

R⁷ is hydrogen, methyl, ethyl, methoxy, or hydroxyl;

Y is substituted or unsubstituted heterocyclo;

R⁹ is aryl or —CH═C(Me)-Aryl; and

W is O or NR⁸ where R⁸ is hydrogen or C₁-C₅ aliphatic, with the provision that when

W is O that Y is not 1,3-dioxolan-1-yl.

In another embodiment of the invention, compounds of the formula IIB are provided wherein:

R¹ is methyl or ethyl;

R⁵ and R⁶ are hydrogen, or R⁵ and R⁶ together form a carbon-carbon bond;

R⁷ is hydrogen, methyl, ethyl, methoxy, or OH;

Y is selected from the group consisting of

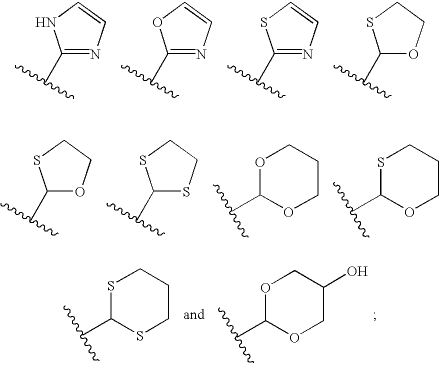

R⁹ is selected from the group consisting of

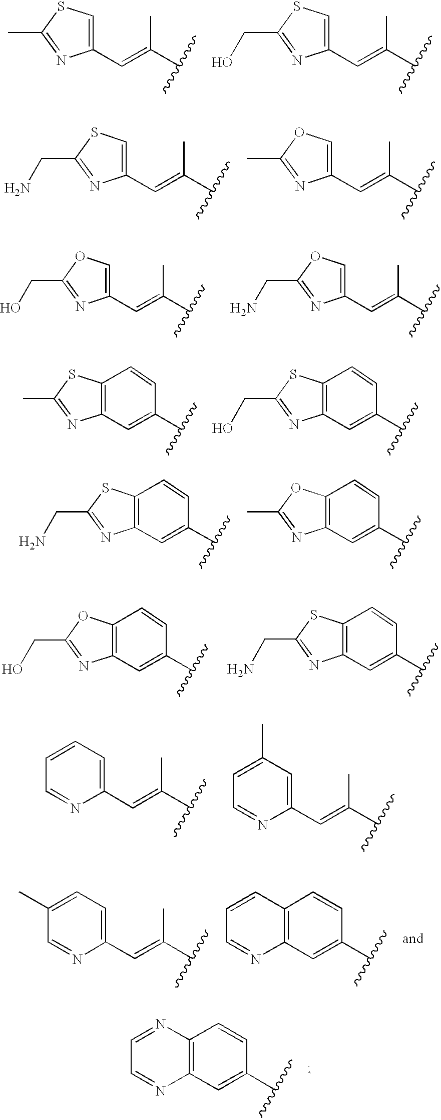

and

W is O or NH, with the proviso that when W is 0 that Y is not 1,3-dioxolan-1-yl.

In another embodiment of the present invention, compounds of the formula IIB are provided

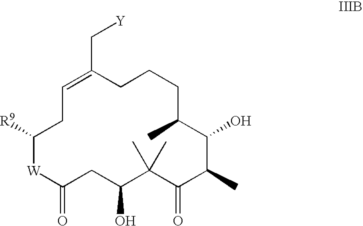

wherein:

Y is selected from the group consisting of

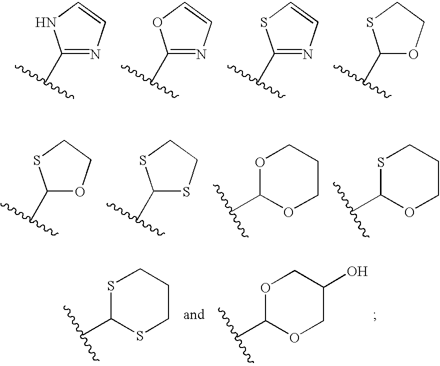

R⁹ is selected from the group consisting of

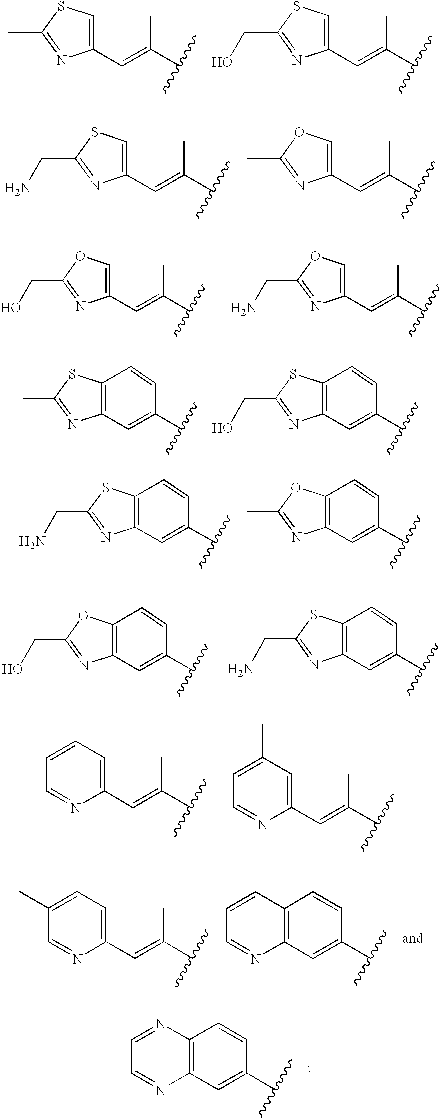

and

W is O or NH.

In another embodiment of the invention, compounds of formula IIIB are provided wherein:

Y is selected from the group consisting of

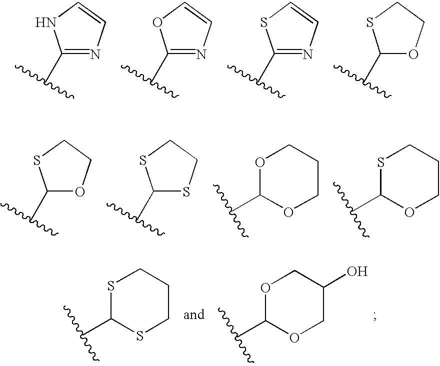

R⁹ is

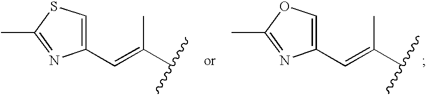

and

W is O or NH.

In another embodiment of the invention, compounds of formula IIIB having the formulas

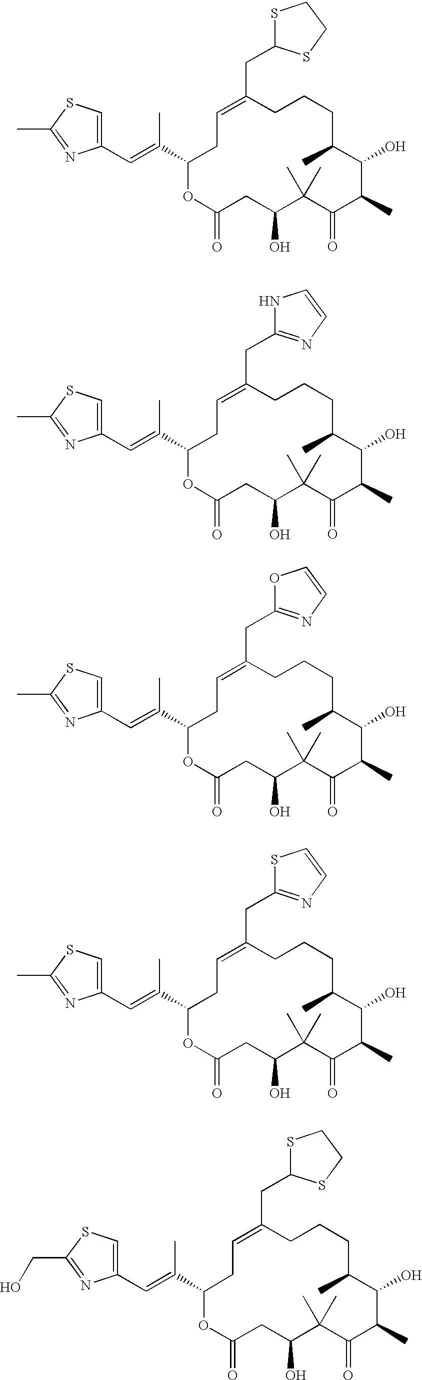

are provided.

The compounds of the present invention are cytotoxic agents and may be used in any suitable manner including but not limited to as anti-cancer agents. An illustrative assay for assessing the degree of cytotoxicity and tubulin polymerization is described in Example 1.

Starting Materials

The inventive methods can be used with epothilone D and with any other naturally occurring epothilone compounds having a methyl at C-12 and a double bond between C-12 and C-13. For example, PCT Publication WO 99/65913 (which is incorporated herein by reference in its entirety) describes 39 naturally occurring epothilones obtained from Sorangium cellulosum So ce 90, many of which are suitable for the practice of the present invention. Examples of suitable epothilones described by WO 99/65913 include: epothilone H₂, epothilone D₁, epothilone D₂, epothilone D₅, epothilone I₃, epothilone I₅, epothilone I₆, and epothilone K. Naturally occurring epothilones possessing a methyl at C-12 and an epoxide between C-12 and C-13 can be converted into their double bond counterparts using a deoxygenation method described by PCT Publication No. WO/99/43653 which is incorporated herein by reference. Briefly, the deoxygenation method comprises reacting the epoxy-containing epothilones with a zinc/copper couple typically in the present of a polar solvent such as isopropanol and water.

Deposits of Sorangium cellulosum strain So ce90 from which epothilones were first extracted exist at the German Collection of Microorganisms as DSM 6773 (PCT publication WO 93/10121) and DSM 11999 (PCT publication WO 99/42602), a mutated version of DSM 6773 which allegedly displays increased production of epothilones A and B over the wild type strain. Fermentation conditions for Sorangium can be based on the protocols described in U.S. Pat. No. 6,194,181, PCT Publication Nos. 93/10121, 97/19086, 98/22461, and 99/42602 and a publication by Gerth et al., 1996, The Journal of Antibiotics, 49: 560-563, each of which is incorporated herein by reference.

Naturally occurring epothilones that are suitable for the practice of the present invention and their derivatives can also be obtained from heterologous host cells using recombinant methods. Procedures for making epothilones in heterologous hosts such as Myxococcus xanthus, Steptomyces lividans, and Pseudomonas fluorescens are described in U.S. Pat. No. 6,303,342 which is incorporated herein by reference. Among other things, the patent provides the nucleotide sequence of the epothilone PKS and modification enzyme genes cloned from Sorangium cellulosum SMP44; cosmids containing overlapping fragments of the epothilone PKS and modification enzyme genes; plasmid pairs having the full complement of epoA, epoB, epoC, epoD, epoE, epoF, epoK, and epoL genes; and heterologous host cells for making epothilones and epothilone derivatives. Cosmids, pKOS35-70.1A2 (ATCC 203782), pKOS35-70.4 (ATCC 203781), pKOS35-70.8A3 (ATCC 203783), and pKOS35-79.85 (ATCC 203780); plasmid pair, pKOS039-124R (PTA-926) and pKOS039-126R (PTA-927); and strain K111-32.25 (PTA-1700) derived from Myxococcus xanthus containing all the epothilone genes and their promoters, have been deposited with the American Type Culture Collection (“ATCC”), Manassas, Va., USA. Additional procedures for making epothilones and epotholone derivatives in Myxococcus xanthus are described in U.S. Ser. No. 09/560,367 filed Apr. 28, 2000, and PCT Publication No. WO 01/83800, entitled PRODUCTION OF POLYKETIDES, both of which are also incorporated herein by reference.

In addition, suitable epothilone compounds can also be obtained from de novo chemical synthesis as described for example by: U.S. Pat. Nos. 6,156,905, 6,043,372 and 5,969,145 and PCT publications WO 98/08849, WO 98/25929, WO 99/01124 each of which is incorporated herein by reference. Additional synthetic methods for making epothilone compounds are described in PCT publications: 97/19086; 98/38192; 99/02514, 99/07692; 99/27890; 99/28324; 99/43653; 99/54318; 99/54319; 99/54330; 99/58534; 99/59985; 99/67252; 99/67253; 00/00485; 00/23452; 00/37473; 00/47584; 00/50423; 00/57874; 00/58254; 00/66589; 00/71521; 01/07439; and 01/27308, each of which is incorporated herein by reference.

Synthetic Methods

General principles of organic chemistry including functional moieties and reactivity and common protocols are described by for example in Advanced Organic Chemistry 3rd Ed. by Jerry March (1985) which is incorporated herein by reference in its entirety. In addition, it will be appreciated by one of ordinary skill in the art that the synthetic methods described herein may use a variety of protecting groups whether or not they are explicitly described. A “protecting group” as used herein means a moiety used to block functional moiety such as oxygen, sulfur, or nitrogen so that a reaction can be carried out selectively at another reactive site in a multifunctional compound. General principles including specific functional groups and their uses are described for example in T. H. Greene and P. G. M. Wuts, Protective Groups in Organic Synthesis, 3rd edition, John Wiley & Sons, New York (1999).

The bicyclo-compounds of the present invention are made from 16-membered-macrocyclic compounds having an aldehyde at C-12 and a carbon-carbon double bond between C-12 and C-13. Compounds having an aldehyde at C-12 can be made from compounds having a hydroxymethyl group at C-12, which in turn can be made from compounds having a methyl group at C-12. Scheme 1 illustrates one embodiment for obtaining compounds having a hydroxymethyl at C-12 from compounds having a methyl group at C-12.

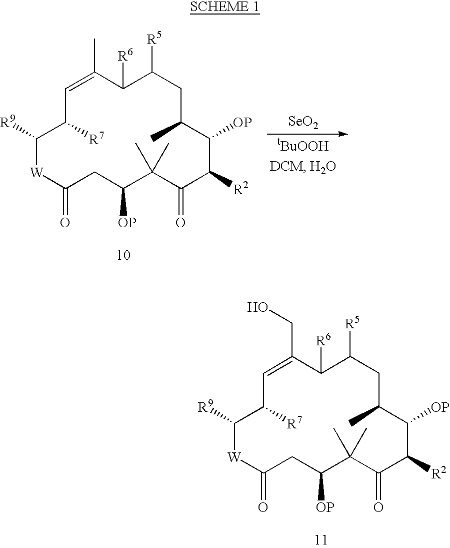

As shown in Scheme 1, treatment of desoxyepothilone 10 (where R², R⁵, R⁶, R⁷, R⁹, and W are as previously described and P is either hydrogen or a hydroxy protecting group) with selenium dioxide under the described conditions results in the C-12 hydroxymethyl (also referred to as C-26 hydroxy) compound 11 along with other products. Under certain reaction conditions (particularly longer reaction times), a 11,26-bis(hydroxy) derivative of compound 10 is also formed. Under other reaction conditions, the 26-oxo derivative of compound 10 is formed directly.

10,11-Dehydroepothilone D can also be obtained M. xanthus strains that make epothilones. For example, K111-72-4.4 expresses the epothilone polyketide synthase and contains an epoK gene with an inactivating in-frame deletion. Strain K111-72-4.4 (PTA-2713) was deposited in the ATCC on Nov. 21, 2000. See also U.S. Ser. No. 09/825,876 filed on Apr. 3, 2001 entitled EPOTHILONE COMPOUNDS AND METHODS OF MAKING AND USING THE SAME by inventors Robert Arslanian, John Carney and Brian Metcalf which is incorporated herein by reference. Hydroxylation of 10,11-dehydroepothilone D using the method described in Scheme 1 provides the corresponding C-12 hydroxymethyl compound for use in the present invention.

Compounds having a hydroxymethyl group at C-12 are converted into compounds having an aldehyde at C-12 by selective oxidation. Scheme 2 illustrates one embodiment of this method.

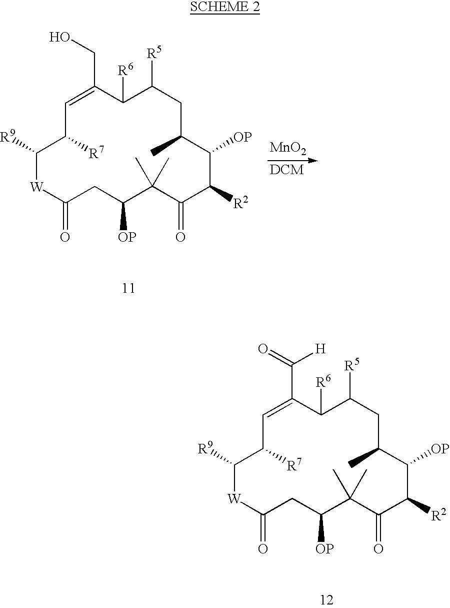

As illustrated in Scheme 2, a compound 11 having a hydroxymethyl at C-12 is oxidized using activated manganese dioxide to a compound 12 having an aldehyde at C-12. Compound 12 can then used to make the bicyclo-compounds of the present invention.

In one aspect of the present invention, a compound having an aldehyde at C-12 is converted into a bicyclic compound 13 having an isoxazoline or a pyrazoline at C-12 and C-13. Scheme 3 illustrates one embodiment of this method.

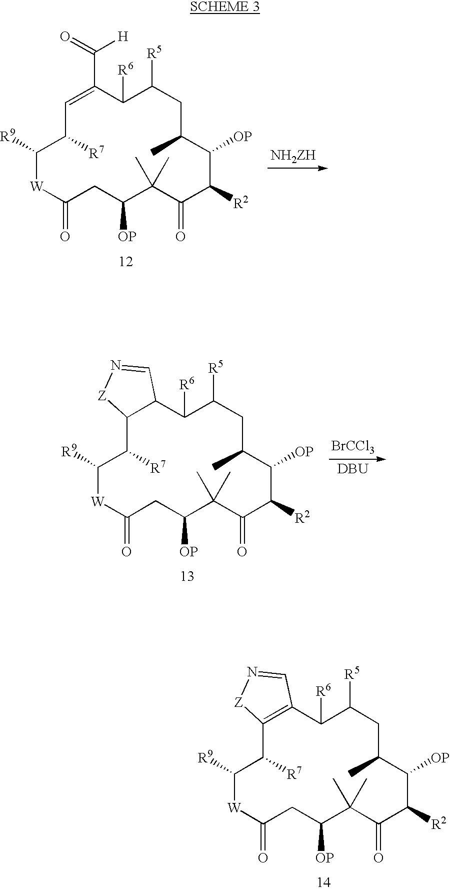

As shown in Scheme 3, compound 12 is treated NH₂ZH, where Z is O, S, NH, or NR′, to yield the corresponding compound 13 having a five-membered heterocyclic moiety fused to C-12 and C-13 of the 16-membered-macrocycle. Compound 13 can optionally be oxidized with an oxidizing agent such as bromotrichloromethane and 1,8-diazabicyclo[5.4.0]undec-7-ene (“DBU”) to yield a compound 14 having an aromatic five membered ring (also referred to as a heteroaryl). When Z is O, the method involves treating compound 12 with hydroxylamine to yield an isoxazoline-containing compound 13 and then optionally an isoxazole-containing compound 14. When Y is NH or NR′ where R′ is C₁-C₁₀ aliphatic or aryl, the method involves treating compound 12 with a hydrazine to yield a pyrazoline-containing compound 13 which then can be optionally converted to a pyrazole-containing compound 14.

In another aspect of the present invention, a compound 12 having an aldehyde at C-12 is converted into a bicyclic compound having a dihydropyrimidine 15 at C-12 and C-13. Scheme 4 illustrates one embodiment of this method.

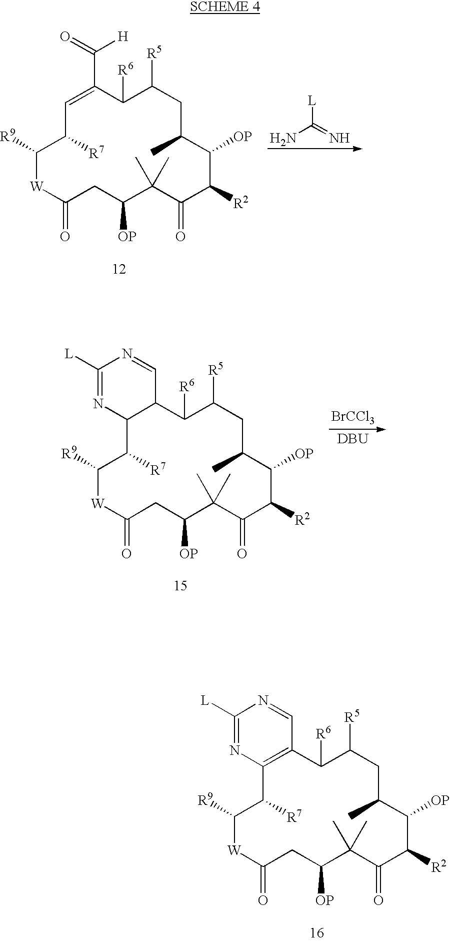

As shown in Scheme 4, compound 12 is treated with NH₂C(═NH)L where L is hydrogen, R′, OR′, NHR′, NR′R″, or SR′ (where R′ and R″ are each independently C₁-C₁₀ aliphatic or aryl), to yield the corresponding compound 15 having a six-membered heterocyclic moiety fused to C-12 and C-13 of the 16-membered macrocycle. Compound 15 can optionally be oxidized to yield a pyrimidine-containing compound 16.

In another aspect of the present invention, a compound 12 having an aldehyde at C-12 is converted into a bicyclic compound 17 having a dihydropyrimidinone or an aminodihydropyrimidine at C-12 and C-13. Scheme 5 illustrates one embodiment of this method.

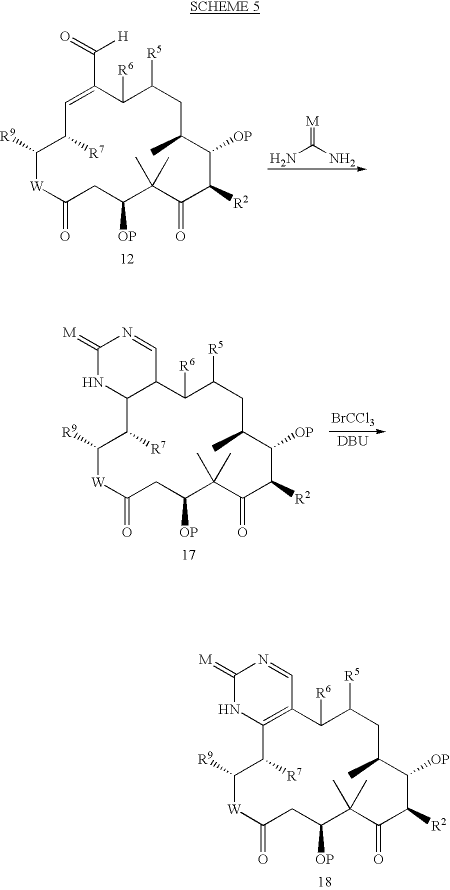

As shown in Scheme 6, compound 12 is treated NH₂C(═M) NH₂ where M is O, S, or NR′ to yield the corresponding compound 17 having a six-membered heterocyclic moiety fused to C-12 and C-13 of the 16-membered-macrocycle. Compound 17 can optionally be oxidized with an oxidizing agent such as bromotrichloromethane and DBU to yield a six membered heteroaryl 18. When Y is O, the method involves treating compound 12 with a urea to yield a dihydropyrimidinone-containing compound 17 which then can be optionally converted to a pyrimidinone- or hydroxypyrimidine-containing compound 18. When Y is S, the method involves treating compound 12 with a thiourea to yield a dihydrothiopyrimidinone-containing compound 17 which then can be optionally converted to a thiopyrimidinone- or mercaptopyrimidine-containing compound 18. When Y is NR′ where R′ C₁-C₁₀ aliphatic or aryl, the method involves treating compound 12 with a guanidine to yield an aminohydropyrimidine-containing compound 17 which then optionally can be converted into an aminopyridine-containing compound 18.

In another aspect of the present invention, compounds where R⁹ is

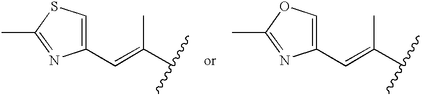

are hydroxylated using a microbially-derived hydroxylase to make the corresponding C-21 hydroxy compounds where R⁹ is

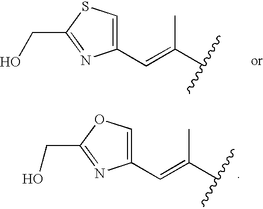

Protocols for effecting such a transformation are described for example by PCT Publication No. WO 00/39276 which is incorporated herein in its entirety by reference, and by Example 18 and 19 herein.

In another embodiment, macrolactones can be converted into the corresponding macrolactams for use as starting material in the practice of the present invention. In another embodiment, inventive macrolactones can be converted into the corresponding macrolactams which are also compounds of the present invention. Scheme 6 illustrates one embodiment where a macrolactone is converted into a suitable macrolactam for the practice of the present invention.

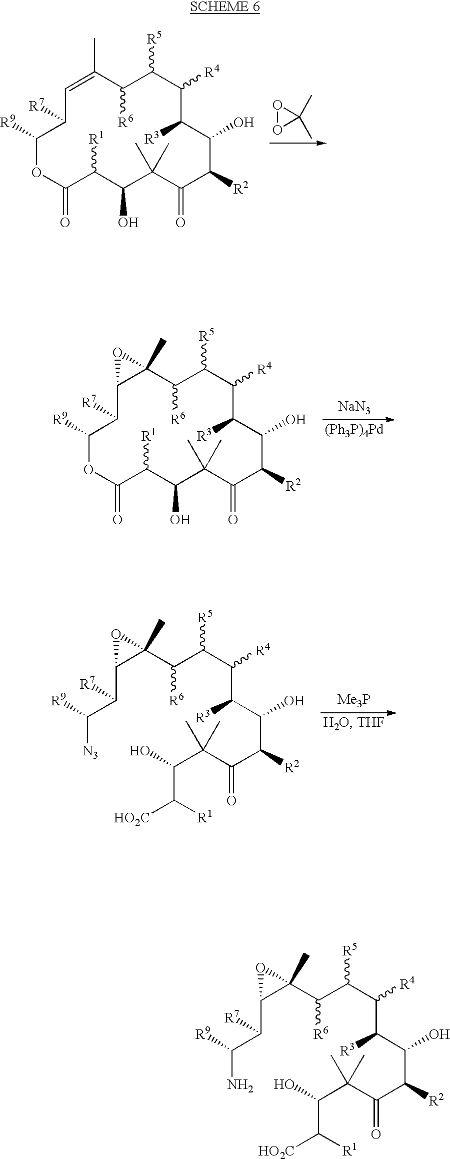

As illustrated by Scheme 6, a desoxy macrolactone is epoxidized using dimethyldioxirane to provided the 12,13-epoxy counterpart. The epoxymacrolactone is treated with sodium azide and tetrakis(triphenylphosphine)palladium to open the ring and form the azido acid. The azide is then reduced with trimethylphosphine in the presence of water to form the amino carboxy acid.

Epoxy-compounds where W is NH can be by the macrolactamization of the amino acid as illustrated in Scheme 7.

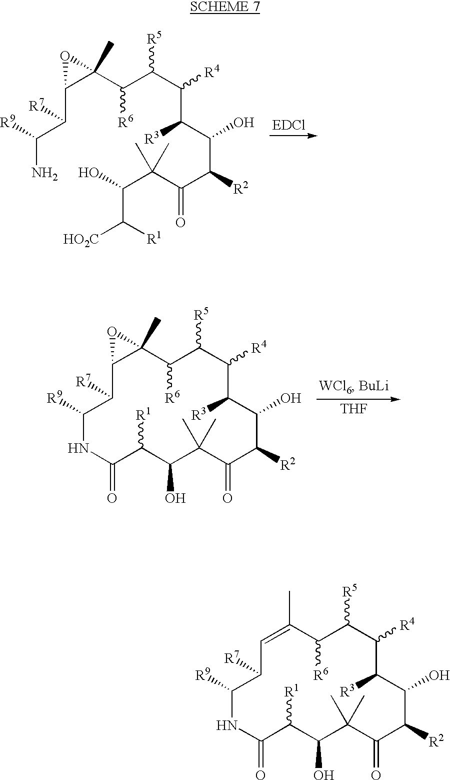

As shown by Scheme 7 the amino carboxy acid is treated with a condensing agent such as 1-(3-dimethylaminopropyl)-3-ethyl-carbodiimide and 1-hydroxybenzotriazole to form the epoxy-macrolactam. The desoxy-macrolactam is made by treating the epoxy-macrolactam with the reagent formed from tungsten hexachloride and butyllithium.

Epoxy-compounds where W is NR⁸ and R⁸ is not hydrogen can be made by treating the amino carboxy acid with an aldehyde or a ketone and sodium cyanoborohydride prior to macrolactamization as illustrated in Scheme 8.

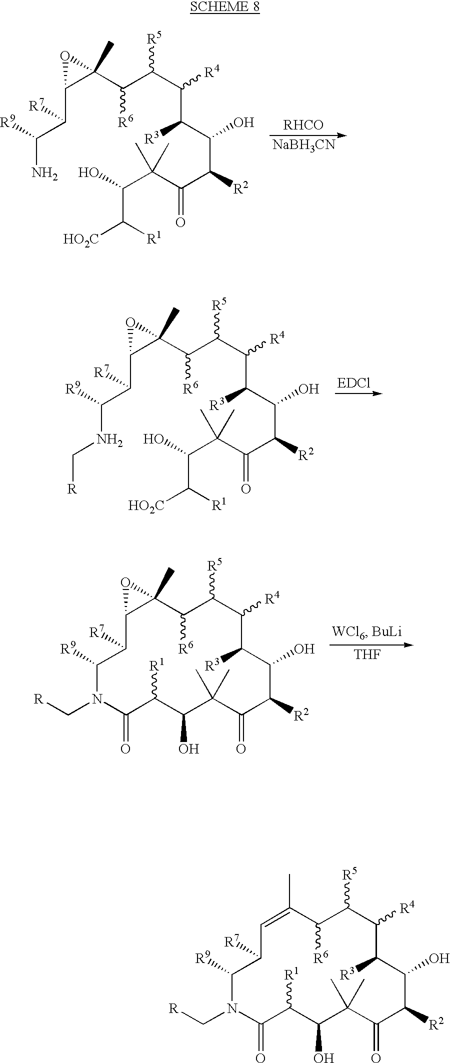

As shown in Scheme 8 the amino carboxy acid is treated with aldehyde RCHO and sodium cyanoborohydride to form a substituted amino carboxy acid which is then macrolactamized. The epoxy compounds can be deoxygenated using tungsten hexachloride and butyllithium.

The synthetic methods for making the macrolactams of the invention are also described in greater detail by the Examples 20-24. Example 20 describes the formation of the amino acid using 9-oxo-epothilone D as an illustrative starting material. Examples 21 and 22 describe the formation of the epoxy and desoxy macrolactam versions of 9-oxo-epothilone D respectively. Examples 23 and 24 describe the formation of the epoxy and desoxy substituted macrolactam versions of 9-oxo-epothilone D respectively.

Compounds of formula IB may be prepared starting from the 26-hydroxy compounds 11 and/or 26-oxo compounds 12. As illustrated in Scheme 9, compounds of formula IB wherein Y=2-thiazolyl may be prepared by reaction of the hydroxy-protected 26-iodo compound with the 2-lithiothiazole followed by deprotection.

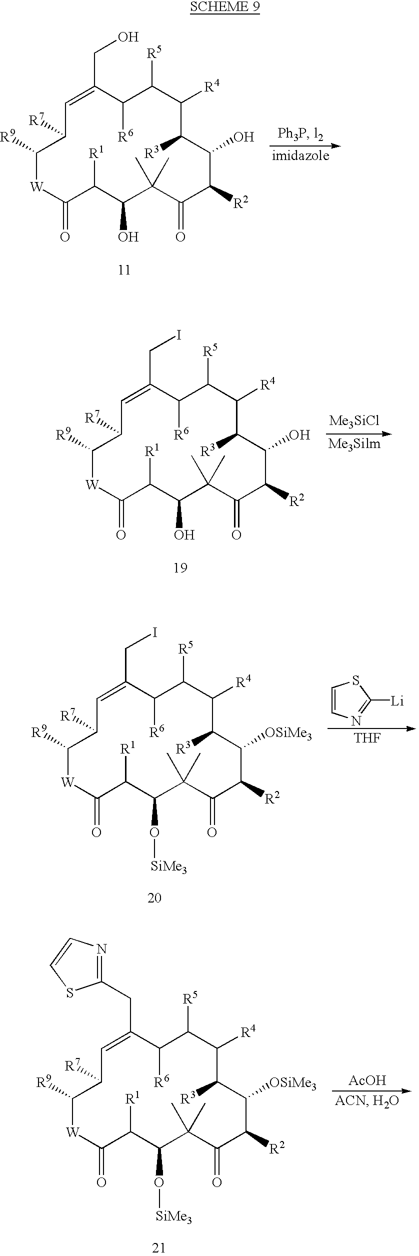

Reaction of 11 with iodine and triphenylphosphine in the presence of imidazole produces the 26-iodide, 19. The hydroxyl groups are then protected, for example using a trialkylsilyl ether such as trimethylsilyl, by reaction with trimethylsilylchloride and trimethylsilylimidazole. The iodide is displaced with the lithium anion of thiazole, produced by reaction of thiazole with butyllithium. The hydroxyl groups are then removed by treatment with acetic acid in aqueous acetonitrile to produce compound 22, the compound of formula IB wherein Y=2-thiazolyl. Compounds of formula IB wherein Y=1,3-dithian-2-yl are prepared from compound 20 in a similar fashion, substituting 2-lithio-1,3-dithiane for 2-lithiothiazole. This procedure is detailed below in Example 20.

Scheme 10 illustrates a method for preparation of a compound of formula IB wherein Y=2-imidazolyl, beginning with 26-oxo compound 12.

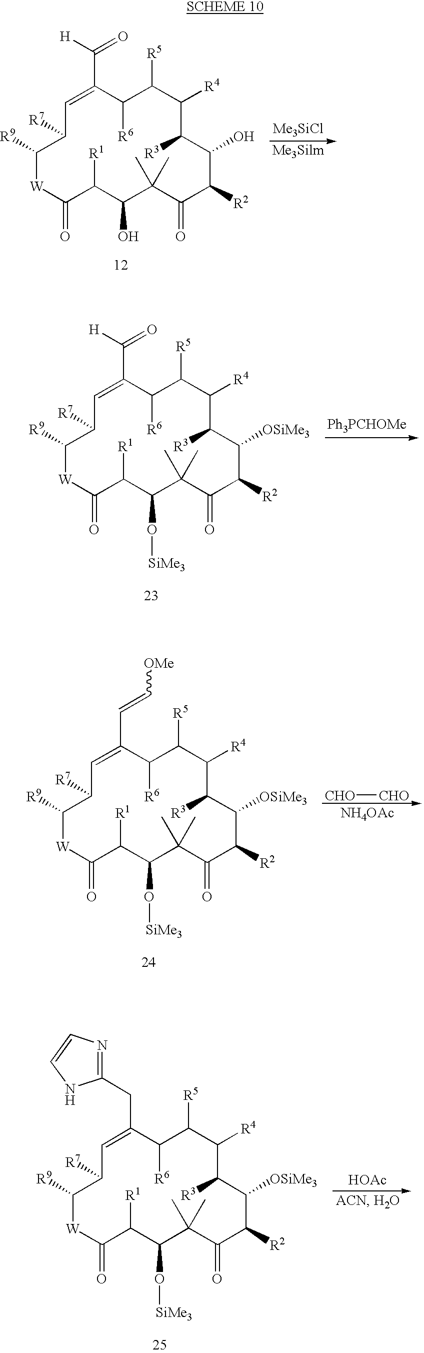

Reaction of compound 12 with trimethylsilylchloride and trimethylsilylimidazole produces the hydroxy-protected compound 23, which is reacted with (methoxymethylidene)triphenylphosphorane to produce the vinyl ether 24. Compound 24 is reacted with aqueous glyoxal and ammonium acetate to form imidazole 25, which is deprotected using acetic acid in aqueous acetonitrile to give compound 26, a compound of formula IB wherein Y=2-imidazolyl.

Formulation

A composition of the present invention generally comprises an inventive compound and a pharmaceutically acceptable carrier. The inventive compound may be free form or where appropriate as pharmaceutically acceptable derivatives such as prodrugs, and salts and esters of the inventive compound.

The composition may be in any suitable form such as solid, semisolid, or liquid form. See Pharmaceutical Dosage Forms and Drug Delivery Systems, 5^{th} edition, Lippicott Williams & Wilkins (1991) which is incorporated herein by reference. In general, the pharmaceutical preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

In one embodiment, the compositions containing an inventive compound are Cremophor®-free. Cremophor® (BASF Aktiengesellschaft) is a polyethoxylated castor oil which is typically used as a surfactant in formulating low soluble drugs. However, because Cremophor® can case allergic reactions in a subject, compositions that minimize or eliminate Cremophor® are preferred. Formulations of epothilone A or B that eliminate Cremophor® are described for example by PCT Publication WO 99/39694 which is incorporated herein by reference and may be adapted for use with the inventive compounds.

Where applicable, the inventive compounds may be formulated as microcapsules and nanoparticles. General protocols are described for example, by Microcapsules and Nanoparticles in Medicine and Pharmacy by Max Donbrow, ed., CRC Press (1992) and by U.S. Pat. Nos. 5,510,118; 5,534,270; and 5,662,883 which are all incorporated herein by reference. By increasing the ratio of surface area to volume, these formulations allow for the oral delivery of compounds that would not otherwise be amenable to oral delivery.

The inventive compounds may also be formulated using other methods that have been previously used for low solubility drugs. For example, the compounds may form emulsions with vitamin E or a PEGylated derivative thereof as described by WO 98/30205 and 00/71163 which are incorporated herein by reference. Typically, the inventive compound is dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration. Another strategy involves encapsulating the inventive compounds in liposomes. Methods for forming liposomes as drug delivery vehicles are well known in the art. Suitable protocols include those described by U.S. Pat. Nos. 5,683,715; 5,415,869, and 5,424,073 which are incorporated herein by reference relating to another relatively low solubility cancer drug taxol and by PCT Publication WO 01/10412 which is incorporated herein by reference relating to epothilone B. Of the various lipids that may be used, particularly preferred lipids for making epothilone-encapsulated liposomes include phosphatidylcholine and polyethyleneglycol-derivitized distearyl phosphatidylethanolamine. Example 25 provides an illustrative protocol for making liposomes containing 9-oxo-epothilone D, the general method which can be readily adapted to make liposomes containing other compounds of the present invention.

Yet another method involves formulating the inventive compounds using polymers such as polymers such as biopolymers or biocompatible (synthetic or naturally occurring) polymers. Biocompatible polymers can be categorized as biodegradable and non-biodegradable. Biodegradable polymers degrade in vivo as a function of chemical composition, method of manufacture, and implant structure. Illustrative examples of synthetic polymers include polyanhydrides, polyhydroxyacids such as polylactic acid, polyglycolic acids and copolymers thereof, polyesters polyamides polyorthoesters and some polyphosphazenes. Illustrative examples of naturally occurring polymers include proteins and polysaccharides such as collagen, hyaluronic acid, albumin, and gelatin.

Another method involves conjugating the compounds of the present invention to a polymer that enhances aqueous solubility. Examples of suitable polymers include polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000 are preferred, with molecular weights between about 20,000 and 80,000 being more preferred and with molecular weights between about 30,000 and 60,000 being most preferred. The polymer is conjugated via an ester linkage to one or more hydroxyls of an inventive epothilone using a protocol as essentially described by U.S. Pat. No. 5,977,163 which is incorporated herein by reference, and by Example 26. Preferred conjugation sites include the hydroxyl off carbon-21 in the case of 21-hydroxy-derivatives of the present invention. Other conjugation sites include the hydroxyl off carbon 3, the hydroxyl off carbon 7 and where applicable, the hydroxyl off carbon 11.

In one embodiment, the compounds of the present invention include a semicarbazide linker which can then be conjugated to targets of interest, including antibodies. The semicarbazide linker is formed by condensing a carbonyl of an inventive compound with a hydrazine derivative. Suitable carbonyl groups include those off carbon-9 (e.g., 9-oxo-epothilone derivatives such as 9-oxo-epothilone D), C-21 (e.g., 21-oxo-epothilone derivatives such as 21-oxo-epothilone D), and C-26 (e.g., 26-oxo-epothilone derivatives such as 26-oxo-epothilone D).

Scheme 11 illustrates one embodiment of a semicarbazide linker using 26-oxo-epothilone D as an example.

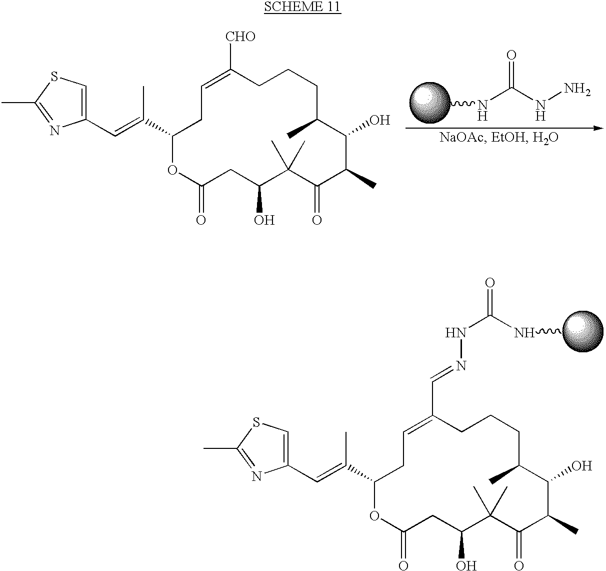

Scheme 12 illustrates another embodiment of a specific semicarbazide linker using 26-oxo-epothilone D as an example.

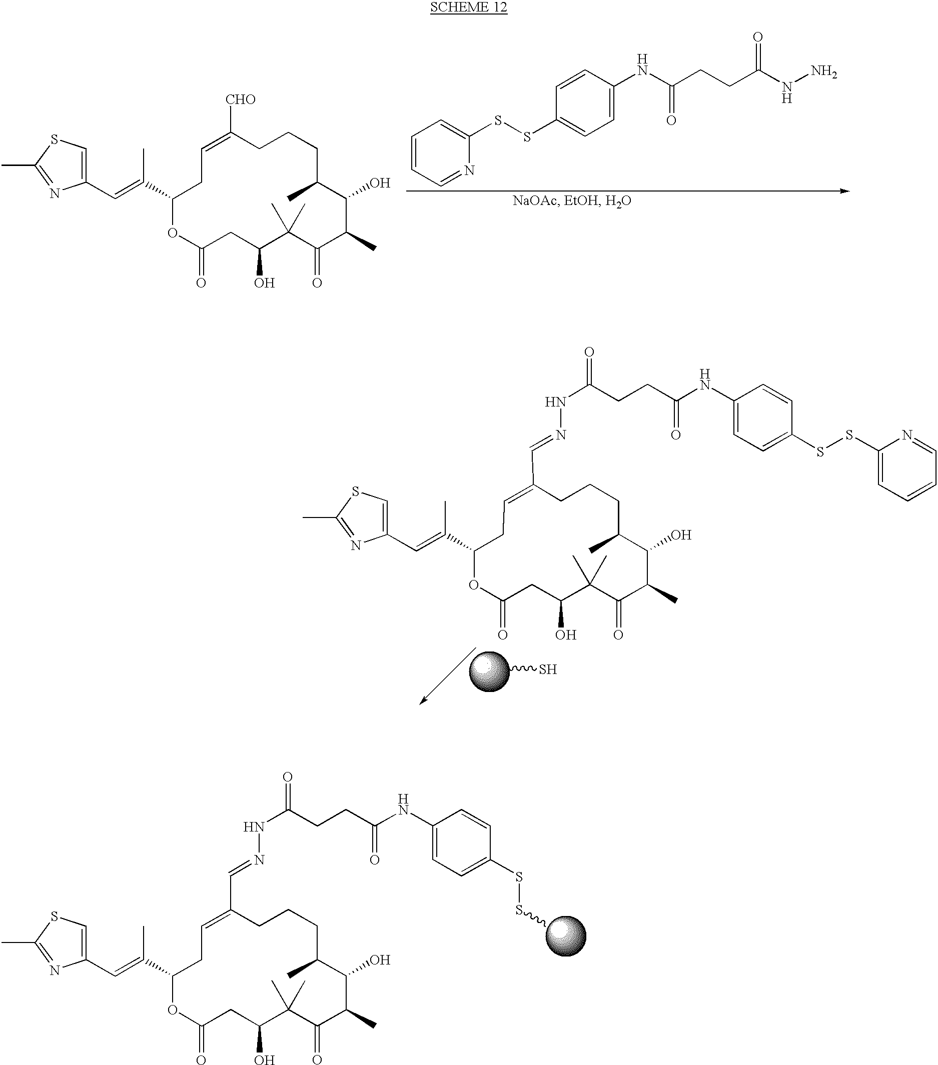

As illustrated by Scheme 10, the semicarbazone-linked epothilone is made and then attached to a target of interest such as an antibody using disulfide exchange.

The amount of active ingredient that may be combined with the carrier materials to produce a single dosage form will vary depending upon the subject treated and the particular mode of administration. For example, a formulation for intravenous use comprises an amount of the inventive compound ranging from about 1 mg/mL to about 25 mg/mL, preferably from about 5 mg/mL to 15 mg/mL, and more preferably about 10 mg/mL. Intravenous formulations are typically diluted between about 2 fold and about 30 fold with normal saline or 5% dextrose solution prior to use.

Methods to Treat Cancer

In one aspect of the present invention, the inventive compounds are used to treat cancer. In one embodiment, the compounds of the present invention are used to treat cancers of the head and neck which include tumors of the head, neck, nasal cavity, paranasal sinuses, nasopharynx, oral cavity, oropharynx, larynx, hypopharynx, salivary glands, and paragangliomas. In another embodiment, the compounds of the present invention are used to treat cancers of the liver and biliary tree, particularly hepatocellular carcinoma. In another embodiment, the compounds of the present invention are used to treat intestinal cancers, particularly colorectal cancer. In another embodiment, the compounds of the present invention are used to treat ovarian cancer. In another embodiment, the compounds of the present invention are used to treat small cell and non-small cell lung cancer. In another embodiment, the compounds of the present invention are used to treat breast cancer. In another embodiment, the compounds of the present invention are used to treat sarcomas which includes fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma. In another embodiment, the compounds of the present invention are used to treat neoplasms of the central nervous systems, particularly brain cancer. In another embodiment, the compounds of the present invention are used to treat lymphomas which include Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma.

The method comprises administering a therapeutically effective amount of an inventive compound to a subject suffering from cancer. The method may be repeated as necessary either to contain (i.e. prevent further growth) or to eliminate the cancer. Clinically, practice of the method will result in a reduction in the size or number of the cancerous growth and/or a reduction in associated symptoms (where applicable). Pathologically, practice of the method will produce at least one of the following: inhibition of cancer cell proliferation, reduction in the size of the cancer or tumor, prevention of further metastasis, and inhibition of tumor angiogenesis.

The compounds and compositions of the present invention can be used in combination therapies. In other words, the inventive compounds and compositions can be administered concurrently with, prior to, or subsequent to one or more other desired therapeutic or medical procedures. The particular combination of therapies and procedures in the combination regimen will take into account compatibility of the therapies and/or procedures and the desired therapeutic effect to be achieved.

In one embodiment, the compounds and compositions of the present invention are used in combination with another anti-cancer agent or procedure. Illustrative examples of other anti-cancer agents include but are not limited to: (i) alkylating drugs such as mechlorethamine, chlorambucil, Cyclophosphamide, Melphalan, Ifosfamide; (ii) antimetabolites such as methotrexate; (iii) microtubule stabilizing agents such as vinblastin, paclitaxel, docetaxel, and discodermolide; (iv) angiogenesis inhibitors; (v) and cytotoxic antibiotics such as doxorubicon (adriamycin), bleomycin, and mitomycin. Illustrative examples of other anti-cancer procedures include: (i) surgery; (ii) radiotherapy; and (iii) photodynamic therapy.

In another embodiment, the compounds and compositions of the present invention are used in combination with an agent or procedure to mitigate potential side effects from the inventive compound or composition such as diarrhea, nausea and vomiting. Diarrhea may be treated with antidiarrheal agents such as opioids (e.g. codeine, diphenoxylate, difenoxin, and loeramide), bismuth subsalicylate, and octreotide. Nausea and vomiting may be treated with antiemetic agents such as dexamethasone, metoclopramide, diphenyhydramine, lorazepam, ondansetron, prochlorperazine, thiethylperazine, and dronabinol. For those compositions that includes polyethoxylated castor oil such as Cremophor®, pretreatment with corticosteroids such as dexamethasone and methylprednisolone and/or H₁ antagonists such as diphenylhydramine HCl and/or H₂ antagonists may be used to mitigate anaphylaxis. Illustrative formulations for intravenous use and pretreatment regiments are described by Examples 27 and 28 respectively.

Methods of Treating of Non-Cancer, Cellular Hyperproliferative Disorders

In another aspect of the present invention, the inventive compounds are used to treat non-cancer disorders that are characterized by cellular hyperproliferation. Illustrative examples of such disorders include but are not limited to: atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis, irritant dermatitis, allergic dermatitis), periodontal disease (i.e., periodontitis), polycystic kidney disease, and type I diabetes.

Other examples include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); and eustachean tube diseases (e.g., strictures of all causes including ideopathic).

The method of treating such diseases comprises administering a therapeutically effective amount of an inventive compound to a subject suffering therefrom. The method may be repeated as necessary. The inventive methods are described in greater detail below with reference to three illustrative non-cancer disorders.

In one embodiment, the compounds of the present invention are used to treat psoriasis, a condition characterized by the cellular hyperproliferation of keratinocytes which builds up on the skin to form elevated, scaly lesions. The method comprises administering a therapeutically effective amount of an inventive compound to a subject suffering from psoriasis. The method may be repeated as necessary either to decrease the number or severity of lesions or to eliminate the lesions. Clinically, practice of the method will result in a reduction in the size or number of skin lesions, diminution of cutaneous symptoms (pain, burning and bleeding of the affected skin) and/or a reduction in associated symptoms (e.g., joint redness, heat, swelling, diarrhea, abdominal pain). Pathologically, practice of the method will result in at least one of the following: inhibition of keratinocyte proliferation, reduction of skin inflammation (for example, by impacting on: attraction and growth factors, antigen presentation, production of reactive oxygen species and matrix metalloproteinases), and inhibition of dermal angiogenesis.

In another embodiment, the compounds of the present invention are used to treat multiple sclerosis, a condition characterized by progressive demyelination in the brain. Although the exact mechanisms involved in the loss of myelin are not understood, there is an increase in astrocyte proliferation and accumulation in the areas of myelin destruction. At these sites, there is macrophage-like activity and increased protease activity which is at least partially responsible for degradation of the myelin sheath. The method comprises administering a therapeutically effective amount of an inventive compound to a subject suffering from multiple sclerosis. The method may be repeated as necessary to inhibit astrocyte proliferation and/or lessen the severity of the loss of motor function and/or prevent or attenuate chronic progression of the disease. Clinically, practice of the method will result in improvement in visual symptoms (visual loss, diplopia), gait disorders (weakness, axial instability, sensory loss, spasticity, hyperreflexia, loss of dexterity), upper extremity dysfunction (weakness, spasticity, sensory loss), bladder dysfunction (urgency, incontinence, hesitancy, incomplete emptying), depression, emotional lability, and cognitive impairment. Pathologically, practice of the method will result in the reduction of one or more of the following, such as myelin loss, breakdown of the blood-brain barrier, perivascular infiltration of mononuclear cells, immunologic abnormalities, gliotic scar formation and astrocyte proliferation, metalloproteinase production, and impaired conduction velocity.

In another embodiment, the compounds of the present invention are used to treat rheumatoid arthritis, a multisystem chronic, relapsing, inflammatory disease that sometimes leads to destruction and ankyiosis of affected joints. Rheumatoid arthritis is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and, eventually the pannus grows to fill the joint space. The pannus develops an extensive network of new blood vessels through the process of angiogenesis that is essential to the evolution of the synovitis. Release of digestive enzymes (matrix metalloproteinases (e.g., collagenase, stromelysin)) and other mediators of the inflammatory process (e.g., hydrogen peroxide, superoxides, lysosomal enzymes, and products of arachadonic acid metabolism) from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis and ultimately bony ankylosis, of the involved joint.

The method comprises administering a therapeutically effective amount of an inventive compound to a subject suffering from rheumatoid arthritis. The method may be repeated as necessary to accomplish to inhibit synoviocyte proliferation and/or lessen the severity of the loss of movement of the affected joints and/or prevent or attenuate chronic progression of the disease. Clinically, practice of the present invention will result in one or more of the following: (i) decrease in the severity of symptoms (pain, swelling and tenderness of affected joints; morning stiffness, weakness, fatigue, anorexia, weight loss); (ii) decrease in the severity of clinical signs of the disease (thickening of the joint capsule. synovial hypertrophy, joint effusion, soft tissue contractures, decreased range of motion, ankylosis and fixed joint deformity); (iii) decrease in the extra-articular manifestations of the disease (rheumatic nodules, vasculitis, pulmonary nodules, interstitial fibrosis, pericarditis, episcleritis, iritis, Felty's syndrome, osteoporosis); (iv) increase in the frequency and duration of disease remission/symptom-free periods; (v) prevention of fixed impairment and disability; and/or (vi) prevention/attenuation of chronic progression of the disease. Pathologically, practice of the present invention will produce at least one of the following: (i) decrease in the inflammatory response; (ii) disruption of the activity of inflammatory cytokines (such as IL-I, TNFa, FGF, VEGF); (iii) inhibition of synoviocyte proliferation; (iv) inhibition of matrix metalloproteinase activity, and/or (v) inhibition of angiogenesis.

In another embodiment, the compounds of the present invention are used to threat atherosclerosis and/or restenosis, particularly in patients whose blockages may be treated with an endovascular stent. Atheroschlerosis is a chronic vascular injury in which some of the normal vascular smooth muscle cells (“VSMC”) in the artery wall, which ordinarily control vascular tone regulating blood flow, change their nature and develop “cancer-like” behavior. These VSMC become abnormally proliferative, secreting substances (growth factors, tissue-degradation enzymes and other proteins) which enable them to invade and spread into the inner vessel lining, blocking blood flow and making that vessel abnormally susceptible to being completely blocked by local blood clotting. Restenosis, the recurrence of stenosis or artery stricture after corrective procedures, is an accelerated form of atherosclerosis.

The method comprises coating a therapeutically effective amount of an inventive compound on a stent and delivering the stent to the diseased artery in a subject suffering from atherosclerosis. Methods for coating a stent with a compound are described for example by U.S. Pat. Nos. 6,156,373 and 6,120, 847. Clinically, practice of the present invention will result in one or more of the following: (i) increased arterial blood flow; (ii) decrease in the severity of clinical signs of the disease; (iii) decrease in the rate of restenosis; or (iv) prevention/attenuation of the chronic progression of atherosclerosis. Pathologically, practice of the present invention will produce at least one of the following at the site of stent implanataion: (i) decrease in the inflammatory response, (ii) inhibition of VSMC secretion of matrix metalloproteinases; (iii) inhibition of smooth muscle cell accumulation; and (iv) inhibition of VSMC phenotypic dedifferentiation.

Dosage Levels

In one embodiment, dosage levels that are administered to a subject suffering from cancer or a non-cancer disorder characterized by cellular proliferation are of the order from about 1 mg/m² to about 200 mg/m² which may be administered as a bolus (in any suitable route of administration) or a continuous infusion (e.g. 1 hour, 3 hours, 6 hours, 24 hours, 48 hours or 72 hours) every week, every two weeks, or every three weeks as needed. It will be understood, however, that the specific dose level for any particular patient depends on a variety of factors. These factors include the activity of the specific compound employed; the age, body weight, general health, sex, and diet of the subject; the time and route of administration and the rate of excretion of the drug; whether a drug combination is employed in the treatment; and the severity of the condition being treated.

In another embodiment, the dosage levels are from about 10 mg/m² to about 150 mg/m², preferably from about 10 to about 75 mg/m² and more preferably from about 15 mg/m² to about 50 mg/m² once every three weeks as needed and as tolerated. In another embodiment, the dosage levels are from about 1 mg to about 150 mg/m², preferably from about 10 mg/m² to about 75 mg/m² and more preferably from about 25 mg/m² to about 50 mg/n² once every two weeks as needed and as tolerated. In another embodiment, the dosage levels are from about 1 mg/m² to about 100 mg/m², preferably from about 5 mg/m² to about 50 mg/m² and more preferably from about 10 mg/m² to about 25 mg/m² once every week as needed and as tolerated. In another embodiment, the dosage levels are from about 0.1 to about 25 mg/m², preferably from about 0.5 to about 15 mg/m² and more preferably from about 1 mg/m² to about 10 mg/m² once daily as needed and tolerated.

A detailed description of the invention having been provided above, the following examples are given for the purpose of illustrating the present invention and shall not be construed as being a limitation on the scope of the invention or claims.

### EXAMPLE 1

Biological Activity

Compounds of the invention are screened for anticancer activity in four different human tumor cell lines (MCF-7 (breast), NCI/ADR-Res (breast, MDR), SF-268 (glioma), NCI-H460 (lung)) using sulforhodamine B (SRB) assay. The cells were maintained in a 5% CO₂-humidified atmosphere at 37 degree in RPMI 1640 medium (Life Technology) supplemented with 10% fetal bovine serum (Hyclone) and 2 mM L-glutamine.

Cytotoxicity of the inventive compounds is determined by SRB assay (Skehan et al., J. Natl. Cancer Inst. 82: 1107-1112 (1990) which is incorporated herein by reference). Cultured cells are trypsinized, counted and diluted to the following concentrations per 100 uL with growth medium: MCF-7, 5000; NCI/ADR-Res, 7500; NCI-H460, 5000; and, SF-268, 7500. The cells are seeded at 100 uL/well in 96-well microtiter plates. Twenty hours later, 100 uL of a compound of interest (ranging from 1000 nM to 0.001 nM diluted in growth medium) is added to each well. After incubation with the compound for 3 days, the cells are fixed with 100 uL of 10% trichloric acid (“TCA”) at 4 degree for 1 hour, and are stained with 0.2% SRB/1% acetic acid at room temperature for 20 minutes. The unbound dye is rinsed away with 1% acetic acid, and the bound SRB is then extracted by 200 uL of 10 mM Tris base. The amount of bound dye is determined by OD 515 nm, which correlates with the total cellular protein contents. The data is then analyzed using Kaleida Graph program and the IC₅₀'s calculated.

For tubulin polymerization assays, MCF-7 cells are grown to confluency in 35 mm-culture dishes and treated with 1 uM of a compound of interest for 0, 1 or 2 hours at 37 degree (Giannakakou et al., J. Biol. Chem. 271:17118-17125 (1997); Int. J. Cancer 75: 57-63 (1998) which are incorporated herein by reference). After washing the cells twice with 2 ml of PBS without calcium or magnesium, the cells are lysed at room temperature for 5-10 minutes with 300 uL of lysis buffer (20 mM Tris, PH 6.8, 1 mM MgCl₂, 2 mM EGTA, 1% Triton X-100, plus protease inhibitors). The cells are scraped and the lysates are transferred to 1.5-ml Eppendof tubes. The lysates are then centrifuged at 18000 g for 12 minutes at room temperature. The supernatant containing soluble or unpolymerized (cytosolic) tubulin are separated from pellets containing insoluble or polymerized (cytoskeletal) tubulin and transferred to new tubes. The pellets are then resuspended in 300 uL of lysis buffer. Changes in tubulin polymerization in the cell are determined by analyzing same volume of aliquots of each sample with SDS-PAGE, followed by immunoblotting using an anti-tubulin antibody (Sigma).

### EXAMPLE 2

Hydroxylation of Epothilone D

A mixture of selenium dioxide (50 mg), 0.4 mL of tert-butylhydroperoxide (5-6 M solution in decane), 0.5 mL of dichloromethane, and 0.35 mL of water was stirred at room temperature for 15 minutes. A solution of epothilone D (200 mg) in 1.5 mL of CH₂Cl₂ was added and the mixture was stirred for 48 hours. The mixture was diluted with 20 mL of CH₂Cl₂ and shaken with 10 mL of sat. aq. NaHCO₃. The phases were separated, and the organic phase was dried over MgSO₄, filtered, and evaporated. The residue was dissolved in 2 mL of CH₂Cl₂ and chromatographed on a 35-gm ISCO silica column equilibrated in 80:20 ethyl acetate/hexanes at a flow rate of 20 mL/min. After 20 minutes, the solvent mixture was ramped to 100% ethyl acetate over a 10 minute period. Products eluted in the following order: (a) unreacted epothilone D (47 mg); (b) mixed carbonyl compounds, including 11-oxo-trans-epothilone D and 26-oxoepothilone D (18 mg); (c) diastereomer B of 11-hydroxy-trans-epothilone D (19 mg); (d) diastereomer A of 11-hydroxy-trans-epothilone D (33 mg); and 26-hydroxyepothilone D (45 mg). 11-hydroxy-trans-epothilone D (diastereomer A): ¹³C-NMR (CDCl₃): δ 220.3, 170.5, 165.0, 152.0, 139.5, 137.2, 122.0, 119.8, 116.0, 78.1, 77.3, 75.3, 71.9, 53.0, 42.2, 38.8, 38.1, 31.7, 31.5,27.8,21.5,19.9,19.0,15.7,15.0,13.9, 11.5. 11-hydroxy-trans-epothilone D (diastereomer B): ¹³C-NMR (CDCl₃): δ 220.0, 170.2, 165.1, 151.9, 140.2, 136.5, 120.9, 119.0, 115.3, 78.8, 77.3, 76.4, 71.5, 52.9, 43.2, 39.3, 36.9, 32.7, 29.6, 28.1, 20.9, 19.7, 18.9, 16.9, 16.5, 14.9, 10.9. 26-hydroxyepothilone D: ¹³C-NMR (CDCl₃): δ 220.6, 170.2, 165.1, 151.8, 141.8, 138.6, 121.6, 119.1, 115.5, 78.1, 74.0, 71.8, 66.1, 53.7, 41.6, 39.6, 37.9, 31.8, 31.6, 27.9, 25.2, 22.8, 18.9, 17.6, 16.0, 15.8, 13.3.

### EXAMPLE 3

3,7-bis(O-triethylsilyl)epothilone D

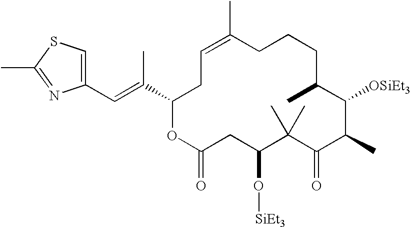

A mixture of epothilone D (50 mg) in CH₂Cl₂ (5 mL), chlorotriethylsilane (100 uL), and 4-dimethylaminopyridine (50 mg) was stirred 12 hours at ambient temperature. The mixture was diluted with ether (25 mL) and washed successively with water, 1 N HCl, sat. NaHCO₃, and brine, then dried over MgSO₄, filtered, and evaporated. The residue was dissolved in hexanes and chromatographed on SiO₂ (hexanes followed by 3:1 hexanes/ether) to yield 62 mg (85%) of product. ¹³C-NMR (CDCl₃): δ 215.3, 171.0, 164.5, 152.6, 140.5, 138.7, 119.6, 119.2, 116.1,79.84,79.83, 76.1,53.5,48.0,39.3, 37.3, 32.4, 32.0, 31.2, 27.4, 24.5, 23.7, 23.0, 19.2, 17.5, 15.0, 7.2 (3C), 6.9 (3C), 5.6 (3C), 5.2 (3C).

### EXAMPLE 4

Hydroxylation of 3,7-bis(O-triethylsilyl)epothilone D

A mixture of selenium dioxide (15 mg), 0.1 mL of tert-butylhydroperoxide (5-6 M solution in decane), 0.2 mL of dichloromethane, and 0.1 mL of water was stirred at room temperature for 15 minutes. A solution of 3,7-bis(O-triethylsilyl)epothilone D (50 mg) (Example 4) in 0.5 mL of CH₂Cl₂ was added and the mixture was stirred for 24 hours. An additional 15 mg of selenium dioxide was added, and the reaction is continued an additional 24 hours. The mixture was diluted with 20 mL of CH₂Cl₂ and shaken with 10 mL of sat. aq. NaHCO₃. The phases were separated, and the organic phase was dried over MgSO₄, filtered, and evaporated. The residue was dissolved in 2 mL of CH₂Cl₂ and chromatographed on a 35-gm ISCO silica column (gradient from 5% ethyl acetate/hexanes to 100% ethyl acetate). The products identified included the 3,7-bis(O-triethylsilyl)-derivatives of the 26-oxo, 26-hydroxy, and 11-hydroxyepothilones D. Also identified was 3,7-bis(O-triethylsilyl)-11,26-dihydroxyepothilone D: ¹³C-NMR (CDCl₃): δ 215.1, 170.8, 164.8, 152.3, 143.7, 137.8, 124.5, 120.4, 116.6, 80.0, 79.0, 76.4, 70.7, 65.8, 53.4, 48.4, 39.3, 36.9, 35.0, 32.2, 28.2, 24.8, 23.9, 19.2 (2C), 17.7, 14.8, 7.2 (3C), 6.9 (3C), 5.6 (3C), 5.2 (3C).

### EXAMPLE 5

10,11-dehydro-epothilone D

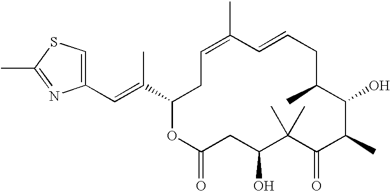

Step 1. 11-(4-toluenesulfonyloxy)-epothilone D

A solution of 11-hydroxy-epothilone D (500 mg) in 10 mL of pyridine and 50 mL of CH₂Cl₂ is cooled on ice and treated with 4-(dimethylamino)pyridine (12 mg) and tosyl chloride (200 mg). After stirring for 4 hours, the mixture is diluted with ethyl acetate and washed sequentially with 1 N HCl, sat. NaHCO₃, and brine, then dried over MgSO₄, filtered, and evaporated. The product is isolated by silica gel chromatography.

Step 2. 10,11-dehydro-epothilone D

A solution of 11-(4-toluenesulfonyloxy)-epothilone D (890 mg) in 10 mL of pyridine is treated with 1,8-diazabicyclo[5.4.0]undec-7-ene (200 mg). The mixture is heated to 90° C. and the reaction is monitored by thin-layer chromatography. When the starting material has disappeared, the mixture is cooled to ambient temperature and concentrated. The residue is dissolved in ethyl acetate and washed sequentially with 1 N HCl, sat. NaHCO₃, and brine, then dried over MgSO₄, filtered, and evaporated. The product is isolated by silica gel chromatography.

### EXAMPLE 6

10,11-dehydro-26-hydroxy-epothilone D

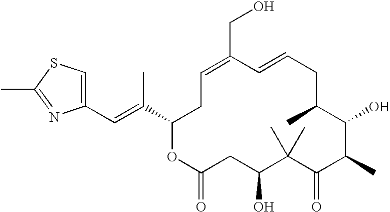

A mixture of selenium dioxide (50 mg), 0.4 mL of tert-butylhydroperoxide (5-6 M solution in decane), 0.5 mL of dichloromethane, and 0.35 mL of water is stirred at room temperature for 15 minutes. A solution of 10,11-dehydroepothilone D (200 mg) in 1.5 mL of CH₂Cl₂ is added and the mixture is stirred for 48 hours. The mixture is diluted with 20 mL of CH₂Cl₂ and shaken with 10 mL of sat. aq. NaHCO₃. The phases are separated, and the organic phase is dried over MgSO₄, filtered, and evaporated. The residue is dissolved in 2 mL of CH₂Cl₂ and chromatographed on a 35-gm ISCO silica column to yield the product.

### EXAMPLE 7

26-hydroxy-epothilone D Lactam

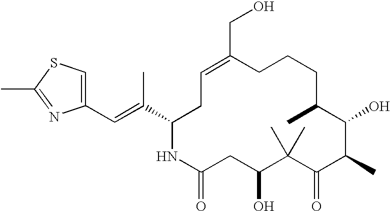

Epothilone D lactam is treated with selenium dioxide according to Example 2. The hydroxylated products are separated by silica gel chromatography.

### EXAMPLE 8

21,26-dihydroxy-epothilone D

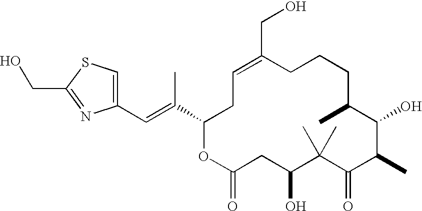

21-hydroxy-epothilone D is treated with selenium dioxide according to Example 2. The hydroxylated products are separated by silica gel chromatography.

### EXAMPLE 9

26-oxo-epothilone D

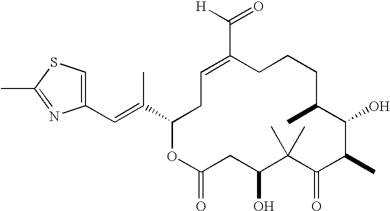

A suspension of 26-hydroxyepothilone D (4 mg) and activated manganese dioxide (16 mg) in 0.2 mL of CH₂Cl₂ was stirred for 1 hour at ambient temperature. The suspension was filtered through a 1-cm plug of silica gel using ethyl acetate, and the eluate was evaporated to yield the product as a clear glass. ¹H-NMR (CDCl₃): δ 9.39 (s,1H), 6.99 (s,1H), 6.45 (br s,1H), 6.45 (dd,1H,J=5.6, 10.0 Hz), 5.41 (dd,1H,J=2.4, 9.6 Hz), 4.30 (m,1H), 3.66 (m,1H), 3.50 (d,1H,J=6.4 Hz), 3.14 (dq,1H,J=4.4, 6.8 Hz), 2.96 (dt,1H,J=9.6, 15.2 Hz), 2.89 (d,1H,J=2.8 Hz), 2.70 (s,3H), 2.64 (ddd,1H,J=2.4,5.2,15.2 Hz), 2.46 (dd,1H,J=10.8, 14.4 Hz), 2.42 (m,1H), 2.26 (dd,1H,J=2.4, 14.4 Hz), 2.21 (dd,1H,J=6.8, 12.8 Hz), 2.13 (d,3H,J=1.2 Hz), 1.70 (m,1H), 1.69 (d,3H,J=1.2 Hz), 1.36 (s,3H), 1.2-1.4 (m,4H), 1.17 (d,3H,J=6.8 Hz), 1.06 (s,3H), 1.00 (d,3H,J=6.8 Hz). ¹³C-NMR (CDCl₃): δ 220.3, 194.7, 170.1, 165.4, 151.6, 148.6, 145.9, 137.9, 120.1, 116.2, 77.3, 73.6, 72.2, 53.6,41.7, 39.6, 37.6, 33.4, 31.4, 25.5, 24.7, 22.9, 19.1, 17.6, 15.9 (2C), 13.0.

### EXAMPLE 10

3,7-bis(O-trimethylsilyl)-26-oxoepothilone D

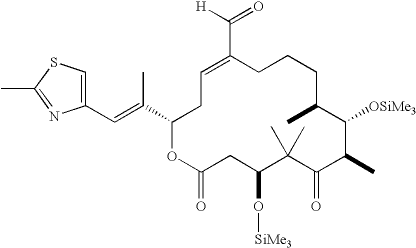

A solution of 26-oxoepothilone D (5.06 g) in 10 mL of CH₂Cl₂ is treated with chlorotrimethylsilane (5.0 mL) and 4-(dimethylamino)pyridine (3.7 g). After stirring for 12 hours, the mixture is diluted with ether and washed sequentially with water and sat. NaHCO₃. The solution is dried over Na₂SO₄, filtered, and evaporated. The product is isolated by flash chromatography on SiO₂.

### EXAMPLE 11

3,7-bis(O-trimethylsilyl)-26-oxoepothilone D Lactam

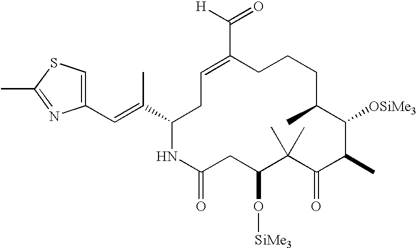

A solution of 26-oxoepothilone D lactam (5.06 g) in 10 mL of CH₂Cl₂ is treated with chlorotrimethylsilane (5 mL) and (4-dimethylamino)pyridine (3.7 g). After stirring for 1 hour, the mixture is diluted with ether and washed sequentially with water and sat. NaHCO₃. The solution is dried over Na₂SO₄, filtered, and evaporated. The product is isolated by flash chromatography on SiO₂.

### EXAMPLE 12

Epothilone D 13,26-isoxazoline

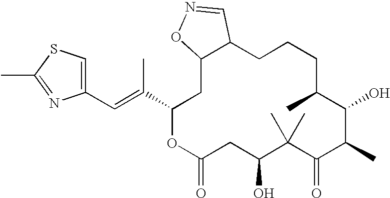

A mixture of 26-oxoepothilone D (500 mg), hydroxylamine hydrochloride (70 mg), and pyridine (2 mL) in 10 mL of ethanol is heated at reflux for 24 hours. The mixture is concentrated, and the residue is dissolved in ethyl acetate. This solution is washed sequentially with 1 N HCl, sat. NaHCO₃, and brine, then dried over MgSO₄, filtered, and evaporated. The resulting crude isoxazoline is purified by silica gel chromatography.

The corresponding pyrazolines are prepared similarly by substituting the appropriate hydrazine in place of hydroxylamine

### EXAMPLE 13

Epothilone D 13,26-isoxazole

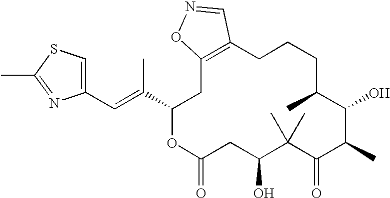

Epothilone D 13,26-isoxazoline (520 mg) is dissolved in 10 mL of 1,2-dichloroethane and treated with bromotrichloromethane (800 mg) and 1,8-diazabicyclo[5.4.0]undec-7-ene (600 mg) at 50° C. for 36 hours. The mixture is cooled to ambient temperature and washed sequentially with 1 N HCl, sat. NaHCO₃, and brine, then dried over MgSO₄, filtered, and evaporated. The product is isolated by silica gel chromatography.

The corresponding pyrazines are prepared by substituting the appropriate pyrazolines in place of the isoxazolines.

### EXAMPLE 14

Epothilone D 13,26-dihydropyrimidine

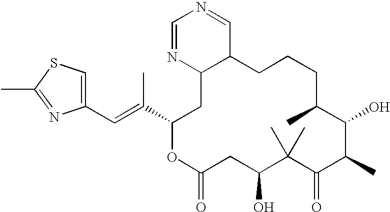

A mixture of 26-oxoepothilone D (500 mg), formamidine hydrochloride (80 mg), and pyridine (2 mL) in 10 mL of ethanol is heated at reflux for 24 hours. The mixture is concentrated, and the residue is dissolved in ethyl acetate. This solution is washed sequentially with 1 N HCl, sat. NaHCO₃, and brine, then dried over MgSO₄, filtered, and evaporated. The resulting dihydropyrimidine is purified by silica gel chromatography.

The corresponding 2-alkoxydihydropyrimidines, 2-(alkylamino)dihydropyrimidines, and 2-thioalkoxydihydropyrimidines are prepared by substituting the formamidine by O-alkylisoureas, N-alkylguanidines, and S-alkylisothioureas, respectively.

### EXAMPLE 15

Epothilone D 13,26-pyrimidine

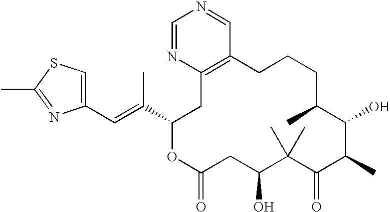

Epothilone D 13,26-dihydropyrimidine (530 mg) is dissolved in 10 mL of 1,2-dichloroethane and treated with bromotrichloromethane (800 mg) and 1,8-diazabicyclo[5.4.0]undec-7-ene (600 mg) at 50° C. for 36 hours. The mixture is cooled to ambient temperature and washed sequentially with 1 N HCl, sat. NaHCO₃, and brine, then dried over MgSO₄, filtered, and evaporated. The product is purified by silica gel chromatography.

The corresponding 2-alkoxypyrimidines, 2-(alkylamino)pyrimidines, and 2-thioalkoxypyrimidines are prepared by substituting the dihydropyrimidine with the 2-alkoxydihydropyrimidines, 2-(alkylamino)dihydropyrimidines, and 2-thioalkoxydihydropyrimidines, respectively.

### EXAMPLE 16

Epothilone D 13,26-(2-aminodihydropyrimidine)

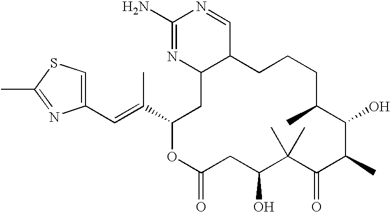

A mixture of 26-oxoepothilone D (500 mg), guanidine hydrochloride (100 mg), and pyridine (2 ml) in 10 ml of ethanol is heated at reflux for 24 hours. The mixture is concentrated, and the residue is dissolved in ethyl acetate. This solution is washed sequentially with 1 N HCl, sat. NaHCO₃, and brine, then dried over MgSO₄, filtered, and evaporated. The product is purified by silica gel chromatography.

The corresponding 13,26-(dihydro-2-pyrimidinone) is prepared by substituting the guanidine with urea in the above procedure.

### EXAMPLE 17

Epothilone D 13,26-(2-aminopyrimidine)

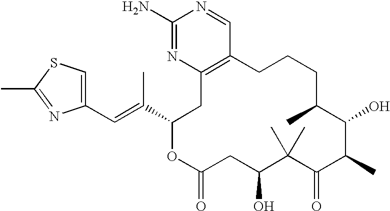

Epothilone D 13,26-(2-aminodihydropyrimidine) (540 mg) is dissolved in 10 mL of 1,2-dichloroethane and treated with bromotrichloromethane (800 mg) and 1,8-diazabicyclo[5.4.0]undec-7-ene (600 mg) at 50° C. for 36 hours. The mixture is cooled to ambient temperature and washed sequentially with 1 N HCl, sat. NaHCO₃, and brine, then dried over MgSO₄, filtered, and evaporated. The product is purified by silica gel chromatography.

The corresponding 13,26-(2-hydroxypyrimidine) is prepared by substituting the (2-aminodihydropyrimidine) with the corresponding 13,26-(dihydro-2-pyrimidinone) in the above procedure.

### EXAMPLE 18

26-(2-dioxolanyl)-epothilone D

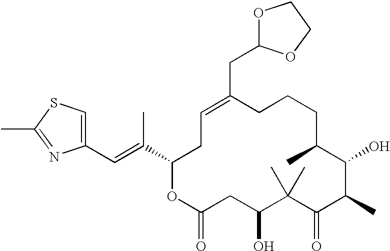

A solution of 3,7-bis(O-triethylsilyl)-26-methoxymethylidene-epothilone D (6.8 g), pyridinium p-toluenesulfonate (0.25 g), and 10 mL of ethylene glycol in 100 mL of THF is stirred at ambient temperature for 12 hours. The mixture is diluted with ethyl acetate and washed successively with water, sat. NaHCO₃, and brine. The solution is dried over Na₂SO₄, filtered, and evaporated. The product is isolated by flash chromatography on SiO₂.

### EXAMPLE 19

26-(5-hydroxy-1,3-dioxan-2-yl)epothilone D

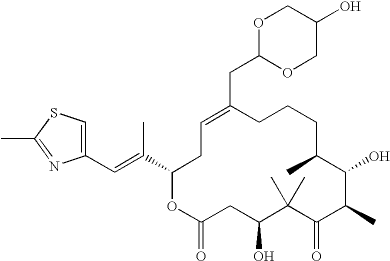

3,7-bis(trimethylsilyl)-26-(methoxymethylene)-epothilone D

A solution of 3,7-bis(O-trimethylsilyl)-26-oxoepothilone D (100 mg) in THF (100 mL) is cooled in a dry ice acetone bath. To this mixture is added a solution of methoxymethylene triphenyl phosphorane in THF (0.25 mmol). The mixture is stirred at bath temperature for 2 h and allowed to warm to room temperature. The mixture is quenched with 100 mL of sat. NaHCO₃ and diluted with ethyl acetate (100 mL). The mixture is shaken and the organic phase separated. The aqueous phase is extracted 2 additional times with 50 mL ethyl acetated. The combined organic phases are then washed with sat'd. NaCl (100 mL) and dried over anhydrous MgSO₄, filtered and concentrated. The residue was dissolved in CH₂Cl₂ (5 mL) and chromatographed (25% ethyl acetated in hexanes) over silica gel to afford 3,7-bis(trimethylsilyl)-26-(methoxymethylene)-epothilone D.

26-(5-hydroxy-1,3-dioxan-2-yl)epothilone D

A solution of 3,7-bis(O-trimethylsilyl)-26-(methoxymethylene)-epothilone D (50 mg) and 1,2,3-propane triol (250 uL) in THF (15 mL) is treated with pyridinium p-toluene sulfonate (15 mg). The mixture is stirred at room temperature for 1 hour. The reaction is quenched by addition of 20 mL of sat. NaHCO₃ and diluted with ethyl acetate (30 mL). The mixture is shaken and the organic phase separated. The aqueous phase is extracted 2 additional times with 20 mL ethyl acetated. The combined organic phases are then washed with sat'd. NaCl (30 mL) and dried over anhydrous MgSO₄, filtered and concentrated. The residue was dissolved in THF (20 mL) and treated with tetra-N-butylamonium fluoride (500 uL, 1.0 M in THF). The mixture is stirred for 12 h. The reaction is quenched by addition of 20 mL of sat. NaHCO₃ and diluted with ethyl acetate (30 mL). The mixture is shaken and the organic phase separated. The aqueous phase is extracted 2 additional times with 20 mL ethyl acetated. The combined organic phases are then washed with sat'd. NaCl (30 mL) and dried over anhydrous MgSO₄, filtered and concentrated. The residue is dissolved in ethyl acetate (1 mL) and chromatographed (25% ethyl acetated in hexanes) over silica gel. Identified from the product mixture was 26-(5-hydroxy-1,3-dioxol-2-yl)epothilone D.

### EXAMPLE 20

### 26-(1,3-dithian-2-yl)epothilone D

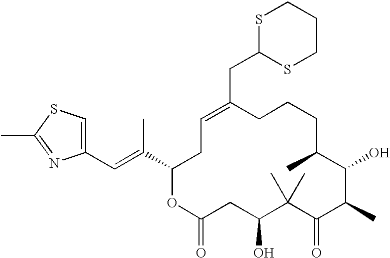

A solution of 3,7-bis(O-trimethylsilyl)-26-hydroxyepothilone D (100 mg) in pyridine (15 mL) is treated with triphenyl phosphine (40 mg) and carbontetrabromide (40 mg). The mixture is stirred at room temperature for 12 h. The mixture is then diluted with ethyl acetate (45 mL) and washed with sat'd. CuSO₄ (2×30 mL) and sat'd NaHCO₃ (45 mL), sat'd NaCl (30 mL), dried over anhydrous MgSO₄, filtered and concentrated. The residue is dissolved in THF (25 mL) and cooled in a dry ice acetone bath. To this cooled mixture is added a solution of 2-lithio-1,3-dithiane in THF (0.2 mmol). The mixture is allowed to warm to room temperature at which point the reaction is quenched by addition of sat'd. NaHCO₃ (25 mL), and ethyl acetate (50 mL). The phases are separated and the organic phase is washed with sat'd. NaCl (20 mL), dried of anhydrous MgSO₄, filtered, and concentrated. The residue was dissolved in THF (20 mL) and treated with tetra-N-butylamonium fluoride (500 uL, 1.0 M in THF). The mixture is stirred for 12 h. The reaction is quenched by addition of 20 mL of sat. NaHCO₃ and diluted with ethyl acetate (30 mL). The mixture is shaken and the organic phase separated. The aqueous phase is extracted 2 additional times with 20 mL ethyl acetate. The combined organic phases are then washed with sat'd. NaCl (30 mL) and dried over anhydrous MgSO₄, filtered and concentrated. The residue is dissolved in ethyl acetate (1 mL) and chromatographed (25% ethyl acetated in hexanes) over silica gel.

Sulfoxide Analog

A solution of 26-(1,3-dithian-2-yl)epothilone D (30 mg) in 95% THF 5% water (10 mL, v/v) is treated with tert-butylhydrogen peroxide (1 equiv). The mixture is stirred for 3 h. The reaction is quenched by addition of saturated ascorbic acid solution (10 ml) and ethyl acetate (20 mL). The mixture is shaken and the organic phase separated. The aqueous phase is extracted 2 additional times with 20 mL ethyl acetate. The combined organic phases are then washed with sat'd. NaCl (30 mL) and dried over anhydrous MgSO₄, filtered and concentrated. The residue is dissolved in ethyl acetate (1 mL) and chromatographed (25% ethyl acetated in hexanes) over silica gel.

### EXAMPLE 21

### 26-(imidazo-2-yl)epothilone D

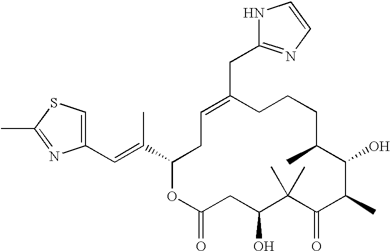

A solution of 3,7-bis(trimethylsilyl)-26-(methoxymethylene)-epothilone D (40 mg) in THF (20 mL) is treated with ammonium acetate (30 mg) and glyoxal (100 uL, 40% w/win water). The mixture is stirred at room temperature for 12 hours. The mixture is treated with sat'd. NaHCO₃ (10 mL) and ethyl acetate (30 mL). The mixture is shaken and the organic phase separated. The aqueous phase is extracted 2 additional times with 15 mL ethyl acetated. The combined organic phases are then washed with sat'd. NaCl (20 mL) and dried over anhydrous MgSO₄, filtered and concentrated. The residue was dissolved in CH₂Cl₂ (2 mL) and chromatographed (25% ethyl acetated in hexanes) over silica gel to afford 26-(imidazo-2-yl)-epothilone D.

### EXAMPLE 22

### 26-(thiazol-2-yl)epothilone D

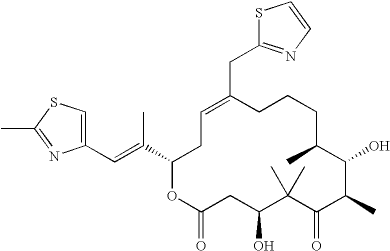

A solution of 3,7-bis(O-trimethylsilyl)-26-hydroxyepothilone D (100 mg) in pyridine (15 mL) is treated with triphenylphosphine (40 mg) and iodine (40 mg). The mixture is stirred at room temperature for 12 h. The mixture is then diluted with ethyl acetate (45 mL) and washed with sat'd. CuSO₄ (2×30 mL) and sat'd NaHCO₃ (45 mL), sat'd NaCl (30 mL), dried over anhydrous MgSO₄, filtered and concentrated. The residue is dissolved in THF (25 mL) and cooled in a dry ice acetone bath. To this cooled mixture is added a solution of 2-lithiothiazole in THF (0.2 mmol). The mixture is allowed to warm to room temperature at which point the reaction is quenched by addition of sat'd. NaHCO₃ (25 mL), and ethyl acetate (50 mL). The phases are separated and the organic phase is washed with sat'd. NaCl (20 mL), dried of anhydrous MgSO₄, filtered, and concentrated. The residue was dissolved in 4:1 THF/water (20 mL) and treated with acetic acid (500 uL). The mixture is stirred for 12 h. The reaction is quenched by addition of 20 mL of sat. NaHCO₃ and diluted with ethyl acetate (30 mL). The mixture is shaken and the organic phase separated. The aqueous phase is extracted 2 additional times with 20 mL ethyl acetate. The combined organic phases are then washed with sat'd. NaCl (30 mL) and dried over anhydrous MgSO₄, filtered and concentrated. The residue is dissolved in ethyl acetate (1 mL) and chromatographed (25% ethyl acetated in hexanes) over silica gel.

### EXAMPLE 23

Microbial Transformation of C-21 Methyl to C-21 Hydroxymethyl

This example describes the microbial transformation of C-21 methyl to C-21 hydroxymethyl of compounds of formula I where Ar is

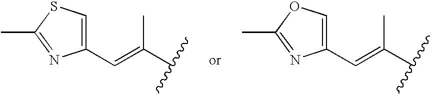

A frozen vial (approximately 2 ml) of Amycolata autotrophica ATCC 35203 or Actinomyces sp. strain PTA-XXX as described by PCT Publication No. WO 00/39276 is used to inoculate 1 500 ml flask containing 100 mL of medium. The vegetative medium consists of 10 g of dextrose, 10 g of malt extract, 10 g of yeast extract, and 1 g of peptone in liter of deionized water. The vegetative culture is incubated for three days at 28° C. on a rotary shaker operating at 250 rpm. One mL of the resulting culture is added to each of sixty-two 500 mL flasks containing the transformation medium which as the same composition as the vegetative medium. The cultures are incubated at 28° C. and 250 rpm for 24 hours. A suitable compound of the invention is dissolved in 155 ml of ethanol and the solution is distributed to the sixty-two flasks. The flasks are then returned to the shaker and incubated for an additional 43 hours at 28° C. and 250 rpm. The reaction culture is then processed to recover 21-hydroxy counterpart of the starting compound.

### EXAMPLE 24

21-hydroxy-epothilone D

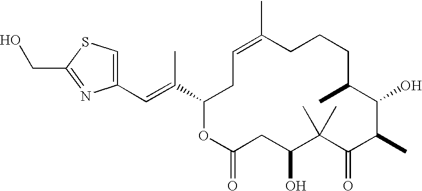

A culture of Sorangium cellulosum So ce90 epoK is grown at 30 C in 8.5 liters of a medium consisting of potato starch (8 g/L), glucose (8 g/L), defatted soybean meal (2 g/L), yeast extract (2 g/L), sodium iron(III)-EDTA (8 mg/L), MgSO₄.7H₂O (1 g/L), CaCl₂-2H₂O (1 g/L), and HEPES buffer (11.5 g/L), adjusted to pH 7.4 using KOH. The culture is stirred at 150 rpm and sparged with sterile air at a rate of 0.1 volumes per minute. After 4 days of growth, the culture is concentrated to a volume of 3 liters by cross-flow filtration across a 0.3-micron membrane. A solution of epothilone D (1 g) in 10 mL of methanol is sterile filtered and added to the concentrated culture. The culture is maintained at 30° C. and is stirred at 450 rpm while sparging with sterile air at a rate of 6 liters per minute. After 24 hours, a 100-mL aliquot of XAD-16 is added to the culture and stirring is continued for an additional hour. The XAD is collected in a filter basket and washed with water to remove culture broth and cells. The XAD is then placed in a chromatography column and eluted with methanol. The eluate is concentrated to an aqueous slurry and then extracted with ethyl acetate. The extract is dried over Na₂SO₄, filtered, and evaporated to yield the crude epothilones. The 21-hydroxyepothilone D is isolated by silica gel chromatography (1:2 hexanes/ethyl acetate).

### EXAMPLE 25

(3S,6R,7S,8R,12R,13S,15S,16E)-15-amino-3,7-dihydroxy-5,9-dioxo-12,13-epoxy-4,4,6,8,12,16-hexamethyl-17-(2-methylthiazol-4-yl)-16-heptadecenoic Acid

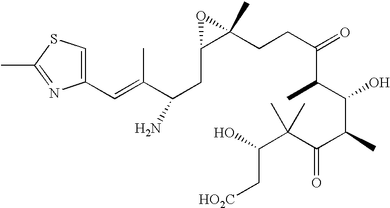

Step 1. 9-oxoepothilone B.

A solution of dimethyldioxirane (0.1 M in acetone, 17 mL) is added dropwise to a solution of 9-oxoepothilone D (505 mg) in 10 mL of CH₂Cl₂ at −78° C. The mixture is warmed to −50° C., kept for 1 hour, and then another portion of dimethyldioxirane solution (5 mL) is added and the reaction is continued for an additional 1.5 hour at −50° C. The reaction is then dried under a stream of N₂ at −50° C. The product is purified by flash chromatography on SiO₂.

Step 2. (3S,6R,7S,8R,12R,13S,15S,16E)-15-azido-3,7-dihydroxy-5.9-dioxo-12,13-epoxy-4,4,6,8,12,16-hexamethyl-17-(2-methylthiazol-4-yl)-16-heptadecenoic acid.

A solution of 9-oxoepothilone B (2.62 g) and sodium azide (0.49 g) in 55 mL of degassed tetrahydrofuran/water (10:1 v/v) is treated with tetrakis(triphenylphosphine)-palladium (0.58 g) under an argon atmosphere. The mixture is kept at 45° C. for 1 hour, then diluted with 50 mL of water and extracted with ethyl acetate. The extract is washed with brine, dried over Na₂SO₄, filtered, and evaporated. The product is purified by flash chromatography on SiO₂.

Step 3. (3S,6R,7S,8R,12R,13S,15S,16E)-15-amino-3,7-dihydroxy-5,9-dioxo-12,13-epoxy-4,4,6,8,12,16-hexamethyl-17-(2-methylthiazol-4-yl)-16-heptadecenoic acid.

A solution of (3S,6R,7S,8R,12R,13S,15S,16E)-15-azido-3,7-dihydroxy-5,9-dioxo-12,13-epoxy-4,4,6,8,12,16-hexamethyl-17-(2-methylthiazol-4-yl)-16-heptadecenoic acid (565 mg) in 15 mL of THF/water (10:1 v/v) is treated with a 1.0 M solution of trimethylphosphine in toluene (3 mL) under argon for 2 hours at ambient temperature. The mixture is concentrated, and the product is purified by flash chromatography on SiO₂.

### EXAMPLE 26

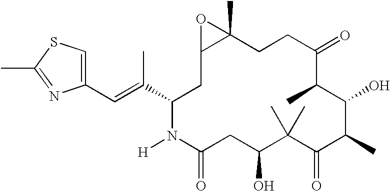

(4S,7R,8S,9R,13R,14S,16S)-13,14-epoxy-4,8-dihydroxy-2,6,10-trioxo-5,5,7,9,13-pentamethyl-16-(1-(2-methylthiazol-4-yl)propen-2-yl)-1-aza-11-cyclohexadecene.

A solution of (3S,6R,7S,8R,12R,13S,15S,16E)-15-amino-3,7-dihydroxy-5,9-dioxo-12,13-epoxy-4,4,6,8,12,16-hexamethyl-17-(2-methylthiazol-4-yl)-16-heptadecenoic acid (540 mg) in acetonitrile/dimethylformamide (20:1 v/v, 150 mL) is cooled to 0° C. and treated sequentially with 1-hydroxybenzotriazole (0.135 g) and 1-(3-dimethylaminopropyl)-3-ethylcarbodiimide hydrochloride (0.5 g). The mixture is warmed to ambient temperature and kept for 12 hours, then diluted with water and extracted with ethyl acetate. The extract is washed sequentially with water, sat. NaHCO₃, and brine, then dried over Na₂SO₄, filtered, and evaporated. The product is purified by flash chromatography on SiO₂.

### EXAMPLE 27

(4S,7R,8S,9R,13Z,16S)-4,8-dihydroxy-26, 10-trioxo-5,5,7,9,13-pentamethyl-16-(1-(2-methylthiazol-4-yl)propen-2-yl)-1-aza-11-cyclohexadecene

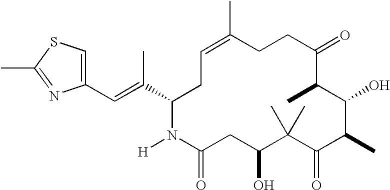

A solution of tungsten hexachloride (0.76 g) in tetrahydrofuran (20 mL) at −78° C. is treated with a 1.6 M solution of n-butyllithium in hexane (2.5 mL). The mixture is allowed to warm to ambient temperature over 20 minutes. A 13.8 mL portion of the resulting green solution is added to a solution of (4S,7R,8S,9R,13R,14S,16S)-4,8-dihydroxy-13,14-epoxy-2,6,10-trioxo-5,5,7,9,13-pentamethyl-16-(1-(2-methylthiazol-4-yl)propen-2-yl)-1-aza-11-cyclohexadecene (360 mg) in 2 mL of tetrahydrofuran at ambient temperature. After 30 min, the reaction is cooled to 0° C. and treated with sat. NaHCO₃ (10 mL). The mixture is diluted with water and extracted with CH₂Cl₂. The extract is dried over Na₂SO₄, filtered, and evaporated. The product is purified by flash chromatography on SiO_{2.}

### EXAMPLE 28

(4S,7R,8S,9R,13R,14S,16S)-13,14-epoxy-4,8-dihdroxy-2,6,10-trioxo-1,5,57,9,13-hexamethyl-16-(1-(2-methylthiazol-4-yl)propen-2-yl)-1-aza-11-cyclohexadecene.

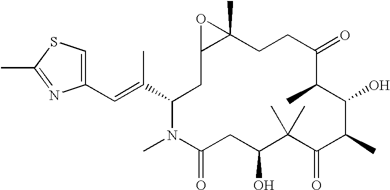

Step 1. (3S,6R,7S,8R,12R,13S,15S,16E)-3,7-dihydroxy-5,9-dioxo-12,13-epoxy-4,4,6,8,12,16-hexamethyl-15-(methylamino)-17-(2-methylthiazol-4-yl)-16-heptadecenoic Acid.

A solution of (3S,6R,7S,8R,12R,13S,15S,16E)-15-amino-3,7-dihydroxy-5,9-dioxo-12,13-epoxy-4,4,6,8,12,16-hexamethyl-17-(2-methylthiazol-4-yl)-16-heptadecenoic acid (540 mg) in 10 mL of methanol is treated with 37% aqueous formaldehyde (1 mL), acetic acid (25 uL), and sodium cyanoborohydride (100 mg). After 1 hour, then mixture is treated with 1N HCl then diluted with ethyl acetate and water. The aqueous phase is extracted with ethyl acetate, and the organic phases are combined, dried over Na₂SO₄, filtered, and evaporated. The product is purified by flash chromatography on SiO₂.

Step 2. (4S,7R,8S,9R,13R,14S,16S)-13,14-epoxy-4,8-dihydroxy-2,6,10-trioxo-1,5,5,7,9,13-hexamethyl-16-(1-(2-methylthiazol-4-yl)propen-2-yl)-1-aza-11-cyclohexadecene.

A solution of (3S,6R,7S,8R,12R,13S,15S,16E)-3,7-dihydroxy-5,9-dioxo-12,13-epoxy-4,4,6,8,12,16-hexamethyl-15-(methylamino)-17-(2-methylthiazol-4-yl)-16-heptadecenoic acid (554 mg) in acetonitrile/dimethylformamide (20:1 v/v, 150 mL) is cooled to 0° C. and treated sequentially with 1-hydroxybenzotriazole (0.135 g) and 1-(3-dimethylaminopropyl)-3-ethylcarbodiimide hydrochloride (0.5 g). The mixture is warmed to ambient temperature and kept for 12 hours, then diluted with water and extracted with ethyl acetate. The extract is washed sequentially with water, sat. NaHCO₃, and brine, then dried over Na₂SO₄, filtered, and evaporated. The product is purified by flash chromatography on SiO₂.

### EXAMPLE 29

(4S,7R,8S,9R,13Z,16S)-4,8-dihydroxy-2,6,10-trioxo-1,5,5,7,9,13-hexamethyl-16-(1-(2-methylthiazol-4-yl)propen-2-yl)-1-aza-11-cyclohexadecene

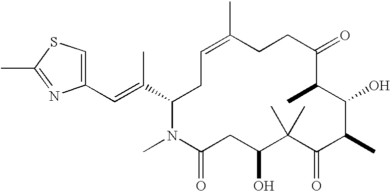

A solution of tungsten hexachloride (0.76 g) in tetrahydrofuran (20 mL) at −78° C. is treated with a 1.6 M solution of n-butyllithium in hexane (2.5 mL). The mixture is allowed to warm to ambient temperature over 20 minutes. A 13.8 mL portion of the resulting green solution is added to a solution of (4S,7R,8S,9R,13R,14S,16S)-13,14-epoxy-4,8-dihydroxy-2,6,10-trioxo-1,5,5,7,9,13-hexamethyl-16-(1-(2-methylthiazol-4-yl)propen-2-yl)-1-aza-11-cyclohexadecene (370 mg) in 2 mL of tetrahydrofuran at ambient temperature. After 30 min, the reaction is cooled to 0° C. and treated with sat. NaHCO₃ (10 mL). The mixture is diluted with water and extracted with CH₂Cl₂. The extract is dried over Na₂SO₄, filtered, and evaporated. The product is purified by flash chromatography on SiO₂.

### EXAMPLE 30

Liposomal Composition

This example describes liposomal compositions containing 9-oxo epothilone. A mixture of lipids and 9-oxo-epothilone D are dissolved in ethanol and the solution is dried as a thin film by rotation under reduced pressure. The resultant lipid film is hydrated by addition of the aqueous phase and the particle size of the epothilone-derivative containing liposomes is adjusted to the desired range. Preferably, the mean particle diameter is less than 10 microns, preferably from about 0.5 to about 4 microns. The particle size may be reduced to the desired level, for example, by using mills (e.g., air-jet mill, ball mill, or vibrator mill), microprecipitation, spray-drying, lyophillization, high-pressure homogenization, recrystrytallization from supercritical media, or by extruding an aqueous suspension of the liposomes through a series of membranes (e.g., polycarbonate membranes) having a selected uniform pore size. In one embodiment, the liposomal composition comprises: an inventive compound (1.00 mg); phosphatidylcholine (16.25 mg); cholesterol (3.75 mg); polyethyleneglycol derivatized distearyl phosphatidylethanolamine (5.00 mg); lactose (80.00 mg); citric acid (4.20 mg); tartaric acid (6.00 mg); NaOH (5.44 mg); water (up to 1 mL). In another embodiment, the liposomal composition comprises: an inventive compound (1.00 mg); phosphatidylcholine (19.80 mg); cholesterol (3.75 mg); distearyl phosphatidylcholine (1.45 mg); lactose (80.00 mg); citric acid (4.20 mg); tartaric acid (6.00 mg); NaOH (5.44 mg); water (up to 1 mL). In yet another embodiment, the liposomal composition comprises: an inventive compound (1.00 mg); 1-palmitoyl-2-oleyl-sn-glycero-3-phosphocholine (17.50 mg); 1-palmitoyl-2-oleyl-sn-glycero-3-phosphoglycerol, Na (7.50 mg); lactose (80.mg); citric acid (4.20 mg); tartaric acid (6.00 mg); NaOH (5.44 mg); water (up to 1 mL). Liposomal compositions containing other compounds of the present invention are prepared using conditions similar to those described above.

### EXAMPLE 31

This example describes the preparation of a poly-glutamic acid-21-hydroxy-9-oxo-epothilone D conjugate. Poly(1-glutamic acid) (“PG”) sodium salt (MW 34 K, Sigma, 0.35 g) is dissolved in water. The pH of the queous solution is adjusted to 2 using 0.2 M HCl. The precipitate is collected, dialyzed against distilled water, and lyophilized to yile 0.29 g of PG. To a solution of PG (75 mg, repeating unit FW 170, 0.44 mmol) in dry DMF (1.5 mL) is added 20 mg of 21-hydroxy-9-oxo-epothilone D, 15 mg of dicyclohexylcarbodiimide (“DCC”) and a trace amount of dimethylaminopyridine. The reaction is allowed to proceed at room temperature for four hours or until completed as indicated by thin layer chromatography. The reaction mixture is poured into chloroform and the resulting precipitate is collected and dried in a vacuum to yield approximately 65 mg of PG-21-hydroxy-9-oxo-epothilone D conjugate. Changing the weight ratio of inventive compound to PG in the starting materials results in polymeric conjugates of various concentrations of 21-hydroxyl-10,11-dehydroepothilone D. Conjugates of other compounds of the present invention are prepared using conditions similar to those described above.

### EXAMPLE 32

Intravenous Formulaion

This example describes an intravenous formuation of 9-oxo-epothilone D. The formulation contains 10 mg/mL of 9-oxo-epothilone D in a vehicle containing 30% propylene glycol, 20% Creomophor EL, and 50% ethanol. The vehicle is prepared by measuring ethanol (591.8 g) to a beaker containing a stir bar; adding Creomophor EL (315.0 g) to the solution and mixing for ten minutes; and then adding propylene glycol (466.2 g) to the solution and mixing for another ten minutes. 9-oxo-epothilone D (1 g) is added to a 1 L volumetric flask containing 400-600 mL of the vehicle and mixed for five minutes. After 10,11-dehydroepothilone D is in solution, the volume is brought to 1 L; allowed to mix for another ten minutes; and filtered through a 0.22 um Millipore Millipak filter. The resulting solution is used to aseptically fill sterile 5 mL vials using a metered peristaltic pump to a targeted fill volume of 5.15 mL/vial. The filled vials are immediately stoppered and crimped.

The vial containing 10 mg/mL of 9-oxo-epothilone D is diluted in normal saline or 5% dextrose solution for administration to patients and administered in non-PVC, non-DEHP bags and administration sets. The product is infused over a one to six hour period to deliver the desired dose.

In one embodiment, the formulation is diluted twenty fold in sterile saline prior to intravenous infusion. The final infusion concentration is 0.5 mg/mL of the inventive compound, 1.5% propylene glycol, 1% Cremophor EL, and 2.5% ethanol which is infused over a one to six hour period to deliver the desired dose.

Intravenous formulations containing other compounds of the present invention may be prepared and used in a similar manner.

### EXAMPLE 33

Pretreatment for Cremophor® Toxicity

This example describes a pretreatment regiment for Cremophor® toxicity. Formulations of a compound of the invention that includes Cremophor® may cause toxicity in patients. Pretreatment with steroids can be used to prevent anaphylaxis. Any suitable corticosterioid or combination of corticosteroid with H₁ antagonists and/or H₂ antagonists may be used. In one embodiment, a subject is premedicated with an oral dose of 50 mg of diphenylhydramine and 300 mg of cimetidine one hour prior to treatment with the inventive compound in a Cremophor® containing formulation. In another embodiment, the subject is premedicated with an intravenous administration of 20 mg of dexamethasone at least one half hour prior to treatment with the inventive compound in a Cremophor® containing formulation. In another embodiment, the subject is premedicated with an intravenous administration of 50 mg of diphenylhydramine, 300 mg of cimetidine and 20 mg of dexamethasone at least one half hour prior to treatment with the inventive compound in a Cremophor® containing formulation. In yet another embodiment, the weight of the subject is taken into account and the subject is pretreated with an administration of diphenylhydramine (5 mg/kg, i.v.); cimetidine (5 mg/kg, i.v).; and dexamethasone (1 mg/kg, i.m.) at least one half hour prior to the treatment with the inventive compound in a Cremophor® containing formulation.

All scientific and patent publications referenced herein are hereby incorporated by reference. The invention having now been described by way of written description and example, those of skill in the art will recognize that the invention can be practiced in a variety of embodiments, that the foregoing description and example is for purposes of illustration and not limitation of the following claims.

What is claimed is:

1. A compound of the formula 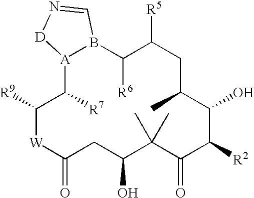
wherein
R² is hydrogen, methyl or ethyl;
R⁵ is hydrogen, oxo, or C₁-C₁₀ aliphatic;
R⁶ is hydrogen, hydroxyl, or oxo, or R⁵ and R⁶ together form a carbon-carbon bond;
R⁷ is hydrogen, methyl, ethyl, methoxy, or hydroxyl;
R⁹ is aryl or —CH═C(Me)-Aryl;
A—B is CH—CH or C═C;
D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl; and
W is O or NR⁸ where R⁸ is hydrogen or C₁-C₅ aliphatic.

2. A compound of claim 1 wherein:
R² is methyl or ethyl;
R⁵ and R⁶ are hydrogen, or R⁵ and R⁶ together form a carbon-carbon bond;
R⁷ is hydrogen, methyl, ethyl, methoxy, or OH;
A—B is CH—CH or C═C;
D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl;
R⁹ is selected from the group consisting of 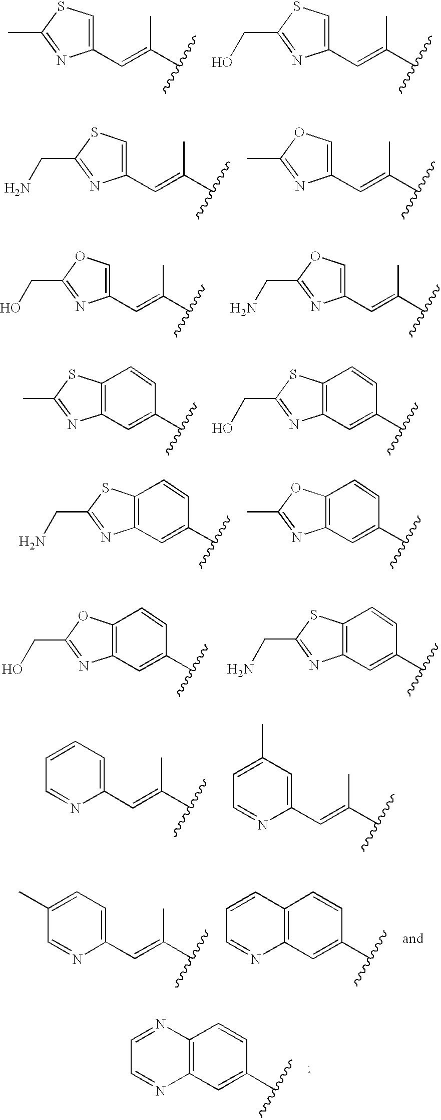
W is O or NH.

3. A compound of claim 1 wherein:
R² is methyl;
R⁵, R⁶, and R⁷ are hydrogen;
A—B is CH—CH or C═C;
D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl;
R⁹ is selected from the group consisting of 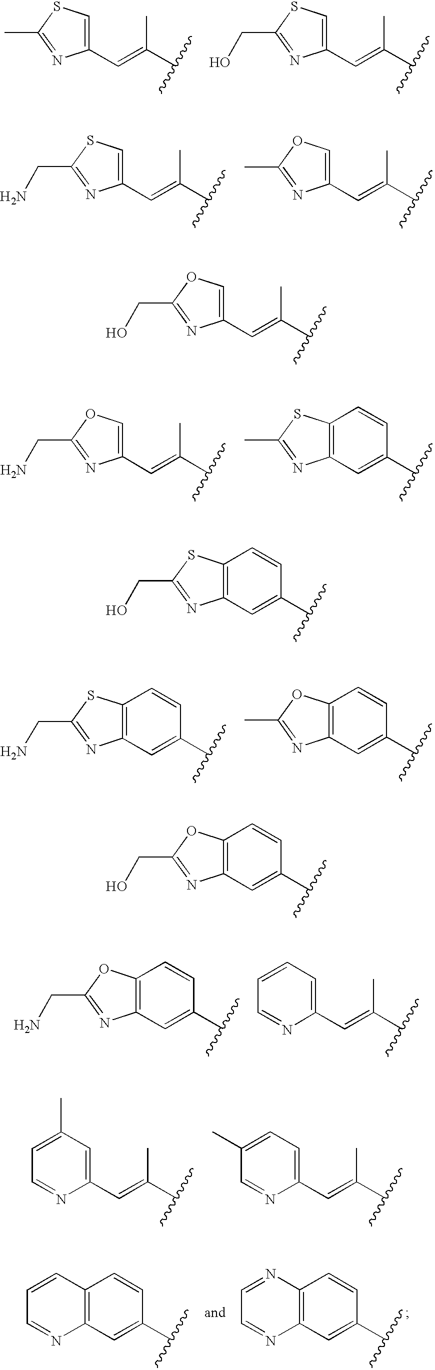
W is O or NH.

4. A compound of claim 1 wherein:
R² is methyl;
R⁵, R⁶, and R⁷ are hydrogen;
A—B is CH—CH or C═C;
D is O, S, NR¹⁰, CR¹⁰═N, C(OR¹⁰)═N, C(NR¹⁰R¹¹)═N, C(SR¹⁰)═N, C(═O)—NH, or C(═NR¹⁰)—NH, where R¹⁰ and R¹¹ are each independently H or C1-C5 aliphatic or aryl;
R⁹ is 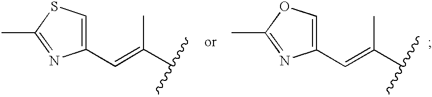
and
W is O or NH.

5. A compound of claim 1 selected from the group consisting of

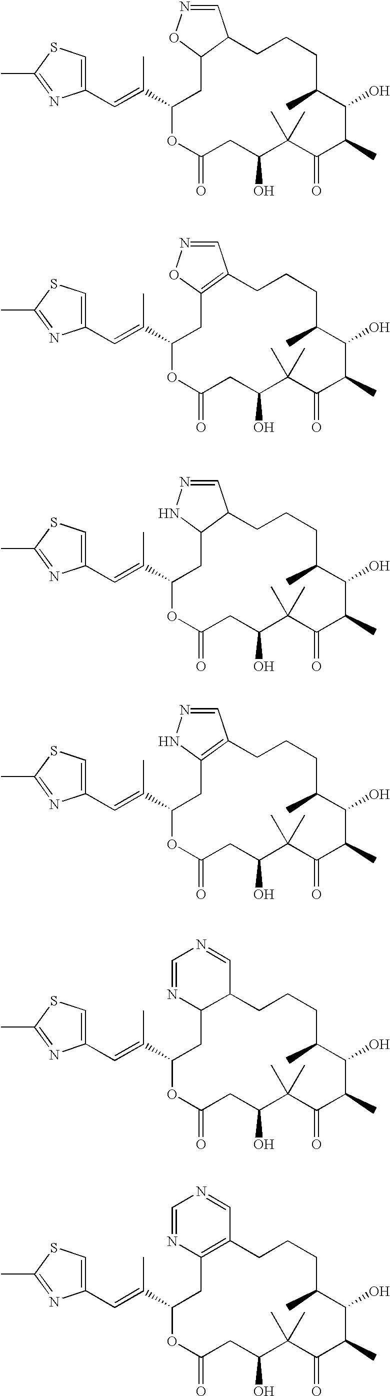

6. A compound of the formula 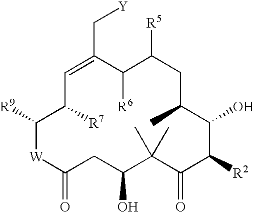
wherein
R² is hydrogen, methyl or ethyl;
R⁵ is hydrogen, oxo, or C₁-C₁₀ aliphatic;
R⁶ is hydrogen, hydroxyl, oxo, or halide, or R⁵ and R⁶ together form a carbon-carbon bond;
R⁷ is hydrogen, methyl, ethyl, methoxy, or hydroxyl;
Y is substituted or unsubstituted heterocyclo;
R⁹ is aryl or —CH═C(Me)-Aryl; and
W is O or NR⁸ where R⁸ is hydrogen or C₁-C₅ aliphatic, with the proviso that when W is O that Y is not 1,3-dioxolan-1-yl.

7. A compound of claim 6 wherein:
R² is methyl or ethyl;
R⁵ and R⁶ are hydrogen, or R⁵ and R⁶ together form a carbon-carbon bond;
R⁷ is hydrogen, methyl, ethyl, methoxy, or OH;
Y is selected from the group consisting of 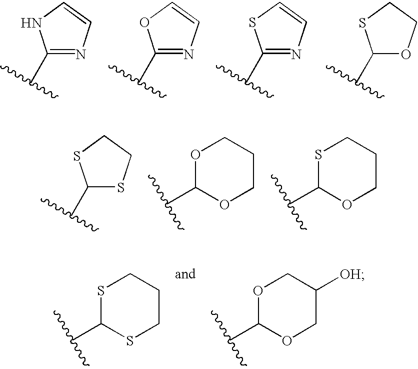
R⁹ is selected from the group consisting of 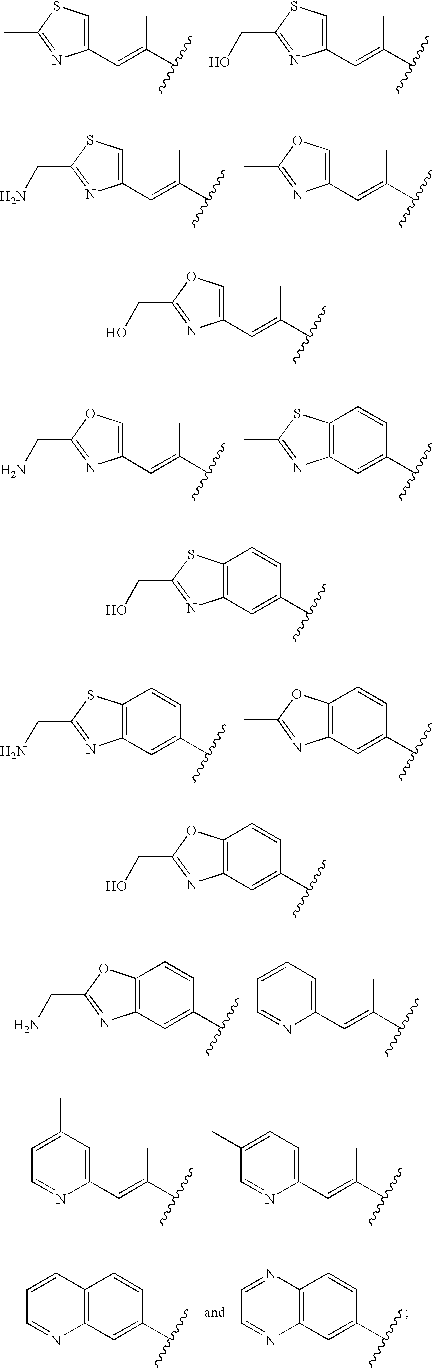
W is O or NH.

8. A compound of claim 6 wherein:
R² is methyl;
R⁵, R⁶, and R⁷ are hydrogen;
Y is selected from the group consisting of 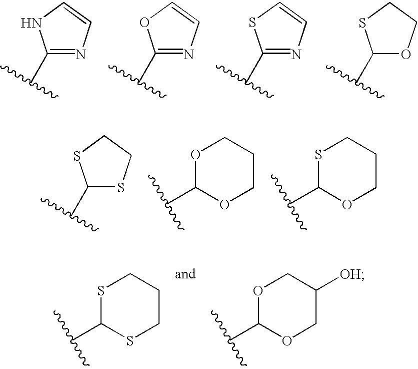
R⁹ is selected from the group consisting of 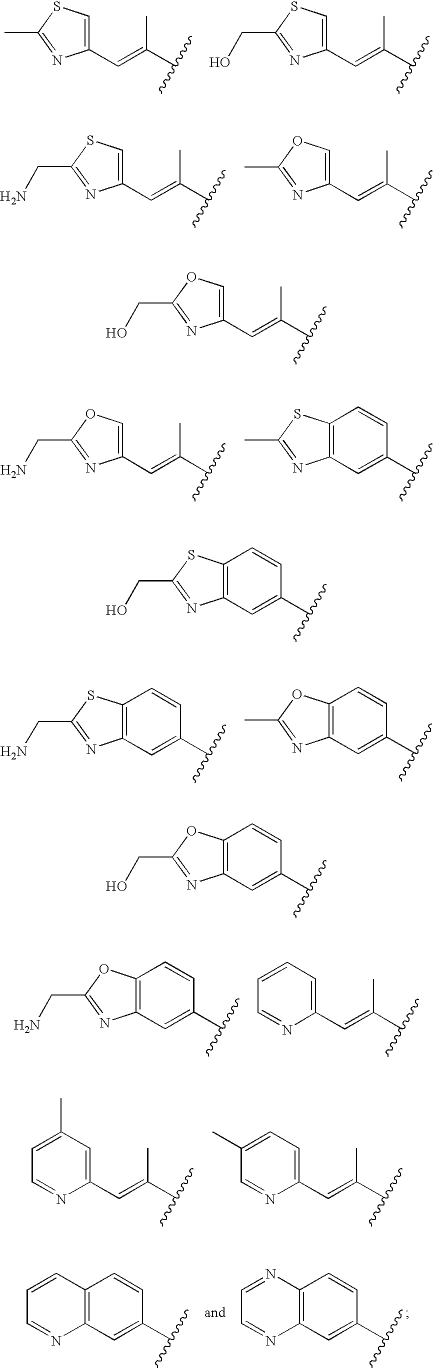
W is or NH.

9. A compound of claim 6 wherein:
R² is methyl;
R⁵, R⁶, and R⁷ are hydrogen;
Y is selected from the group consisting of 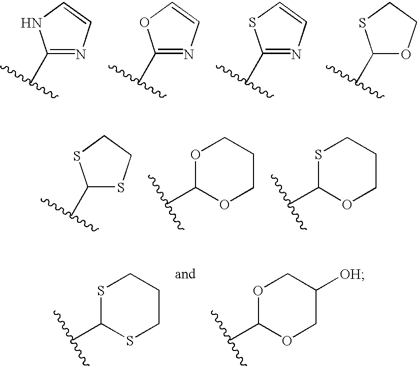
R⁹ is 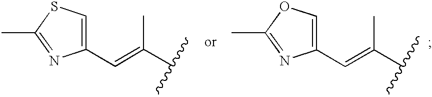
; and
W is O or NH.

10. A compound of claim 6 selected from the group consisting of

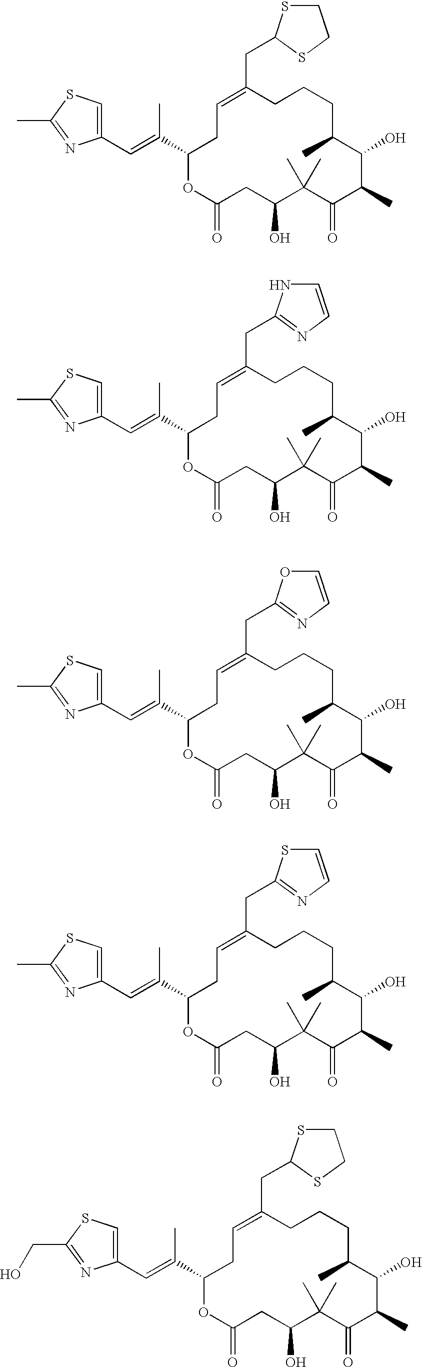

11. The compound of claim 6 having the formula

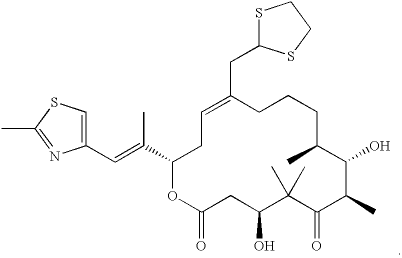

12. The compound of claim 6 having the formula

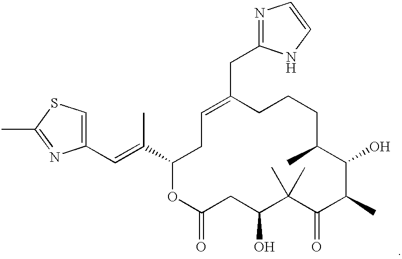

13. The compound of claim 6 having the formula

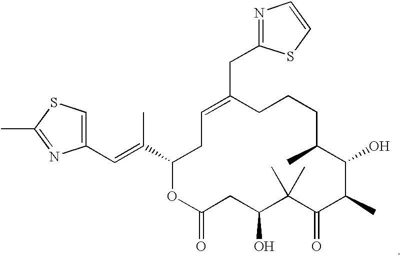

14. The compound of claim 6 having the formula

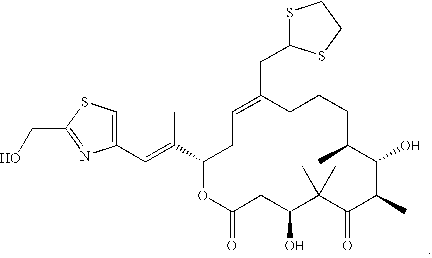

15. The compound of claim 6 having the formula

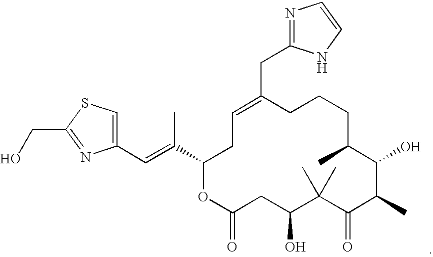

16. The compound of claim 6 having the formula

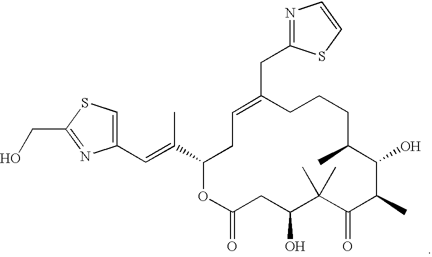

17. A composition comprising a compound of claim 1 optionally admixed with a pharmaceutically acceptable carrier.

18. A method for the treatment of a disease or condition characterized by undesired cellular hyperproliferation in a subject suffering therefrom comprising administering a therapeutically effective amount of a composition of claim 17.

19. The method of claim 18 wherein the disease or condition characterized by undesired cellular hyperproliferation is cancer.

20. The method of claim 18 wherein the disease or condition characterized by undesired cellular hyperproliferation is selected from the group consisting of atrophic gastritis, inflammatory hemolytic anemia, graft rejection, inflammatory neutropenia, bullous pemphigoid, coeliac disease, demyelinating neuropathies, dermatomyositis, inflammatory bowel disease, multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema, periodontal disease, polycystic kidney disease, and type I diabetes.
